(19) United States
(12) Patent Application Publication
(10) Pub. No.: US 2025/0312162 A1
(43) Pub. Date: Oct. 9, 2025

(54) REVERSE SHOULDER SURGERY AND METHOD

(71) Applicant: Arthrosurface Incorporated, Franklin, MA (US)

(72) Inventor: Steven W. EK, Bolton, MA (US)

(73) Assignee: Arthrosurface Incorporated, Franklin, MA (US)

(21) Appl. No.: 19/242,945

(22) Filed: Jun. 18, 2025

Related U.S. Application Data
(63) Continuation of application No. PCT/US2023/084642, filed on Dec. 18, 2023.
(60) Provisional application No. 63/388,000, filed on Dec. 19, 2022.

(51) Int. Cl. A61F 2/40; A61F 2/30; A61F 2/46

(52) U.S. Cl. CPC A61F 2/4014; A61F 2/30734; A61F 2/4081; A61F 2/4684; A61F 2002/30224; A61F 2002/30245; A61F 2002/30331; A61F 2002/4022; A61F 2002/4085; A61F 2002/4659; A61F 2002/4661

(57) ABSTRACT

Described herein are reverse shoulder devices and related systems, methods, and kits useful in implanting a humeral implant in the humerus bone. In some embodiments, a humeral implant device is described that includes a liner, a tray, and an adaptor. In some embodiments, the humeral implant further includes a stem. In some embodiments, the liner is configured to receive a glenosphere. In some embodiments, an offset tool is described that comprises a distal portion configured to interface with a humeral implant stem and a proximal portion configured to interface with a humeral implant tray. In some embodiments, an offset tool is described for determining a desired size of an adapter for use with a humeral implant. In some embodiments, the offset tool includes a trial tray assembly, an outer shaft, and an inner shaft.

### CROSS-REFERENCE

This application claims the benefit of and priority to U.S. Patent Application No. 63/388,000, titled “Reverse Shoulder Surgery and Method”, and filed Dec. 19, 2022, which is incorporated herein by reference in its entirety.

### TECHNICAL FIELD

The present disclosure is related to devices, systems, methods, and kits for reverse shoulder surgery. Specifically, the disclosure is directed to a humeral implant, a glenoid implant, and related instruments for performing surgery to implant such implants.

### BACKGROUND

Articular bones in the human body have articular cartilage covering the ends of the bone, particularly where one bone interfaces with another bone such as in a joint. Articular cartilage is smooth, load bearing, and lubricious, allowing one bone to slip past another bone while maintaining strength during movement. When a bone is injured, this articular cartilage may be damaged. Furthermore, as the body ages articular cartilage can naturally break down, causing bone to rub on bone leading to pain for the patient, reduced mobility, and osteoarthritis.

In some cases, the extent of damage necessitates repair to the cartilage using one or more implants. When replacing a joint with an implant it is important to choose an implant that is mechanically stable and allows for full mobility and movement in the joint.

The implant should be designed to maximize the patient's comfort, minimize damage to surrounding areas, minimize potential further injury, maximize the functional life of the implant, and be easy to install. The longer the amount of time a patient spends in surgery, the greater risk there is for complications for the patient and increased recovery time. Therefore, there is a need for tools, surgical techniques, and systems to reduce overall surgery time.

It is important to preserve as much bone as possible when performing a joint replacement surgery. Joint replacement surgery can require revisions to the placement of an implant or may necessitate an entirely new implant be placed. When placing a new implant or moving an existing implant, the amount of healthy bone for placement of the implant will impact the ease and speed of placement surgery. Without sufficient healthy bone, an implant cannot be placed. Therefore, there is a need for tools, systems, and methods to preserve as much healthy bone as possible when performing orthopedic surgeries.

Traditional joint replacement involves removing the upper condyle or a portion of the upper condyle of an articulating bone. An implant may then be placed into the bone with a stem and cemented into place.

The systems methods and devices described herein are directed towards preserving healthy bone and reducing surgery time for orthopedic surgeries. In some embodiments, the devices, systems, and methods described herein are directed towards reverse shoulder surgery.

### SUMMARY

In one aspect, embodiments described herein relate to systems for performing a reverse shoulder replacement surgery on a patient. In some embodiments, a system for performing a reverse shoulder replacement surgery includes a tray having a body, a lip extending from a proximal portion thereof, and a tray base portion extending from a bottom portion of the body. The body is configured to be at least partially received within a concave inner surface of an implant site in a humerus of the subject. The lip is configured to engage an outer surface of the implant site, thereby coupling the tray to the humerus. The system includes a liner for receiving a head implant and configured to be at least partially disposed within the tray. The system includes a stem configured to be disposed at least partially within the humerus and including an open cavity at a proximal portion thereof configured to receive at least a portion of the tray base portion. The system also includes an adapter disposed between the stem and the tray and configured to at least partially fill a spacing between an inner surface within the cavity of the stem and the tray base portion. In some embodiments, the inner surface of the implant site includes an opening for the tray base portion to extend therethrough towards the stem.

In some embodiments, the adapter includes a foundation portion and a wall extending therefrom. The adaptor foundation portion and the wall define a hollow portion therein configured to at least partially receive the tray base portion. In some embodiments, the adapter is configured with a cylindrical or substantially cylindrical shape. In some embodiments, a desired size of the adapter corresponds to the spacing between the inner surface within the stem cavity and the tray base portion. The desired size of the adapter includes a thickness of the foundation portion, a length of the adapter, a width of the adapter, and/or a thickness of the wall.

In some embodiments, the system further includes an adapter offset tool configured to determine the spacing between the inner surface within the cavity and the tray base portion when inserted therein, so as to determine the desired size of the adapter. In some embodiments, the adapter offset tool includes an outer shaft and an inner shaft. The inner shaft is configured to move telescopically within a channel of the outer shaft. In some embodiments, wherein, when the inner shaft distal portion abuts an inner surface of a cavity of the stem and the outer shaft distal end abuts the trial tray coupled to the implant site, the desired size of the adapter corresponds with a depth therebetween. In some embodiments, an indicator on an outer surface of the distal portion of the inner shaft is visible through a window of the outer shaft indicating the depth of the distal portion within the outer shaft channel.

In some embodiments, the offset tool further includes a trial tray configured to be received by the concave inner surface of the implant site. The trial tray includes an inner surface at a distal portion thereof configured to abut with the outer shaft and enabling the inner shaft to extend from the outer shaft through an opening in the trial tray towards the stem. In some embodiments, the liner, the tray, the adapter, and the stem are stacked together so as to obtain a desired tension provided for a glenohumeral joint of the subject. In some embodiments, the lip and the body of the tray are configured to be secured to the implant site via friction fit. In some embodiments, the tray is detachably coupled to the implant site.

In some embodiments, the stem includes a distal portion extending from the proximal portion. The stem is configured to be disposed within the humerus bone without affixation to the proximal portion. The stem is configured to be secured within the humerus bone via the coupling between the tray and the implant site. In some embodiments, the system includes a glenoid implant disposed in an implant site at a glenoid of the subject. The glenoid implant includes the head implant and is configured to articulate within an inner surface of the liner. In some embodiments, the head implant includes a glenosphere.

In another aspect, an embodiment disclosed herein relates to a method of forming a humeral implant in the humerus of a subject. In some embodiments, the method includes forming an implant site within the humerus. The implant site includes a concave inner surface and an outer surface. The concave inner surface includes an opening at a distal portion. The method also includes the step of inserting a stem at least partially within the humerus. The stem includes an open cavity configured to be contiguous with the opening of the concave inner surface when the stem is disposed within the humerus. The method also includes the step of determining a desired size of an adapter configured to be at least partially disposed within the open cavity. The method also includes the step of securing a tray assembly to the implant site. The tray assembly includes the adapter, a tray, and/or the inner cavity of the implant site, and a liner. The liner is configured to be received at least partially within the tray. The tray is configured to at least partially be received within the open cavity. The liner is configured to at least partially receive a head implant therein. In some embodiments, securing the tray assembly includes providing the adapter, the tray, and the liner as separate components to the implant site or coupled together. In some embodiments, the tray includes a body, a lip extending from a proximal portion thereof, and a tray base portion extend from a bottom portion of the body. The body is configured to be at least partially received within the concave inner surface of the implant site. The lip is configured to engage the outer surface of the implant site, thereby configuring the tray to be secured to the implant site. In some embodiments, the desired size of the adapter is based on a spacing between an inner surface of the cavity and the tray base portion disposed at least partially therein. In some embodiments, determining the desired size of the adapter includes using an offset tool. In some embodiments, forming the implant site includes removing at least a portion of a humeral head to form the outer surface of the implant site. Forming the implant site also includes reaming at least a portion of the humeral head to form the inner cavity surface of the implant site. Forming the implant site also includes forming an opening at a distal portion of the inner cavity surface of the implant site. In some embodiments, removing at least a portion of the humeral head does not form a flat or substantially flat surface between the outer surface of the implant site and the concave inner surface of the implant site.

In another aspect, embodiments disclosed herein relate to an offset tool for determining a desired size of an adapter for use with a humeral implant. In some embodiments, the offset tool includes a trial tray configured to be coupled with a humeral implant site. The trial tray includes an opening at a distal portion thereof; and an outer shaft having a distal end and a proximal end. The distal end includes an aperture to a channel within the outer shaft. The distal end has a dimension larger than a dimension of the opening such that the distal end is configured to abut the trial tray at inner surface thereof. The offset tool also includes an inner shaft having an inner shaft proximal portion and an inner shaft distal portion. The inner shaft proximal portion is configured to move telescopically within the channel of the outer shaft. The inner shaft is configured to extend from the outer shaft and through the opening towards a stem disposed in a humeral stem.

When the inner shaft distal portion abuts an inner surface of a cavity of the stem, the desired size of the adapter corresponds with a depth of the inner shaft proximal portion within the channel of the outer shaft when the distal end of the outer shaft abuts the trial tray coupled to the implant site. In some embodiments, the offset tool further includes at least one groove recessed in the distal end of the distal portion. In some embodiments, the offset tool further includes at least one groove recessed in the distal end of the distal portion. In some embodiments, the offset tool further includes a window disposed through the outer shaft. In some embodiments, the offset tool further includes an indicator on an outer surface of the proximal portion of the inner shaft which is visible through a window of the outer shaft and indicates the depth of the proximal portion within the outer shaft channel. In some embodiments, the offset tool further includes offset markings disposed around the window. In some embodiments, the offset tool further includes an offset indicator disposed on the inner shaft. The offset indicator is configured to be visible through the window.

### BRIEF DESCRIPTION OF THE DRAWINGS

FIGS. 1A-B illustrate an offset tool system, according to an embodiment herein. FIG. 1A illustrates an exploded view of a trial tray and the offset tool system having an outer shaft, an inner shaft, and, according to an embodiment herein. FIG. 1B illustrates the offset tool system from FIG. 1A interfacing with a bone for measuring a depth thereof, according to an embodiment herein.

FIG. 2 illustrates a distal portion of the outer shaft of the offset tool system from FIG. 1A, depicting a measuring tool scale display, according to an embodiment herein.

FIG. 3 illustrates the outer shaft of the offset system from FIG. 1A, according to an embodiment herein.

FIG. 4 illustrates the inner shaft of the offset tool system from FIG. 1A, according to an embodiment herein.

FIGS. 5A-C illustrate a proximal view, a side view, and a distal view, respectively, of the trial tray of the offset tool system from FIG. 1A, according to an embodiment herein.

FIGS. 6A-C illustrate various views of the offset tool system from FIG. 1A interfacing with a bone, according to an embodiment herein.

FIGS. 7A and B illustrate cutaway views an implant tray and adaptor prior to and after interfacing with a humerus bone, according to an embodiment herein.

FIG. 8 illustrates a cutaway view of the tray and adapter from FIG. 7B, further including a liner, according to an embodiment herein.

FIGS. 9A-W illustrate tools, systems, and methods used in relation to a humeral implant in a reverse shoulder surgery, according to an embodiment herein.

FIGS. 10A-R illustrate tools, systems, and methods used in relation to a glenoid implant in a reverse shoulder surgery, according to an embodiment herein.

FIG. 11 illustrates an exploded view of a reverse shoulder system with a humeral implant and a glenoid implant, according to an embodiment herein.

FIGS. 12A-C illustrate views of a baseplate in combination with a fixation screw, according to an embodiment herein.

FIG. 13 illustrates a baseplate at a glenoid implant site, according to an embodiment herein.

### DETAILED DESCRIPTION

This disclosure presents various systems, components, and methods related to a reverse shoulder surgery. Each of the systems, components, and methods disclosed herein provides one or more advantages over traditional systems, components, and methods. Various embodiments of the reverse shoulder surgery devices, systems, components, and methods are disclosed herein.

As used herein, the terms “lines with corresponding numbers” or “tick marks with corresponding numbers” refers to numbers which correspond to one or more line or tick mark. In some embodiments, a line is a substantially straight line.

As used herein, the term “handle” refers to any protuberance or recess from a surface which may be gripped with a hand, tool, or device.

As used herein, the terms “proximal” and “distal” refer to the proximal and distal directions relative to the user of the component. For example, if a surgeon is holding a tool used to place reverse shoulder implant component, the distal and proximal portions of the tool are relative to the surgeon holding the tool. In another example, for components of a glenoid and humeral implant, the distal and proximal portions of the implant components are relative to the patient when the implant has been placed. The term “proximal” refers to an area, surface, or point situated nearer to the center of the body. The term “distal” refers to an area, surface, or point situated further from the center of the body.

Glenoid and/or humoral implants may be placed to add the articulating surfaces of a shoulder joint. Specifically, the glenoid and/or humeral implants are disposed at least partially within the humorous and/or glenoid portion of the scapula bones. Some embodiments described herein are directed to a device used for shoulder surgery where a stem may be free floating, uncemented, or free from a stem altogether.

Disclosed herein, in some aspects, are systems and methods for performing a reverse shoulder surgery. In some embodiments, the system comprises a humeral implant and/or a glenoid implant. In some embodiments, the humeral implant comprises a tray configured to detachably couple to a humeral implant site of a humerus, wherein the tray is configured to receive a liner for receiving a head portion of an implant. In some embodiments, the humeral implant includes a stem at least partially inserted within the humerus. In some embodiments, the stem, at a proximal portion thereof, includes an opening to a cavity within the stem configured to receive a tray base portion. In some embodiments, an adapter is configured to be received by the cavity and is selectively sized so as to interface between an inner surface of the stem defining the cavity and the tray base portion. In some embodiments, the adapter is sized so as to eliminate or reduce unfilled spacing between at least a portion of the cavity inner surface and the tray base portion, thereby eliminating or reducing a potential for movement of the tray and/or stem about the humeral implant site. In some embodiments, the adapter is sized using an offset tool, configured to measure a distance between an opening at the humeral implant site and a bottom inner surface of the cavity.

In some embodiments, aside from the coupling of the stem to the adapter and/or tray, and at least partially due to a friction fit of the stem within the humerus bone, the stem is not otherwise secured to the humerus via additional securing means (e.g., there is no use of bone cement, threading of the stem to the humerus bone, additional screws, etc.). Accordingly in some embodiments, the humeral implant provides only one means of fixation to the humerus (via the tray engagement with the humerus implant site as described herein). In some embodiments, such single means of fixation provides flexibility for the installation and/or arrangement of the humerus implant, as well as flexibility with the interaction with the glenoid implant. Accordingly, in some embodiments, for such single means of fixation (via the tray engagement, for example), the humerus implant does not and is not provided with a stem as described herein. Thus, for such cases where a stem is not included, a reduced amount of bone is removed from the humerus as compared with traditional reverse shoulder replacement surgeries, where installation of the stem would require a corresponding amount of humerus bone to be removed.

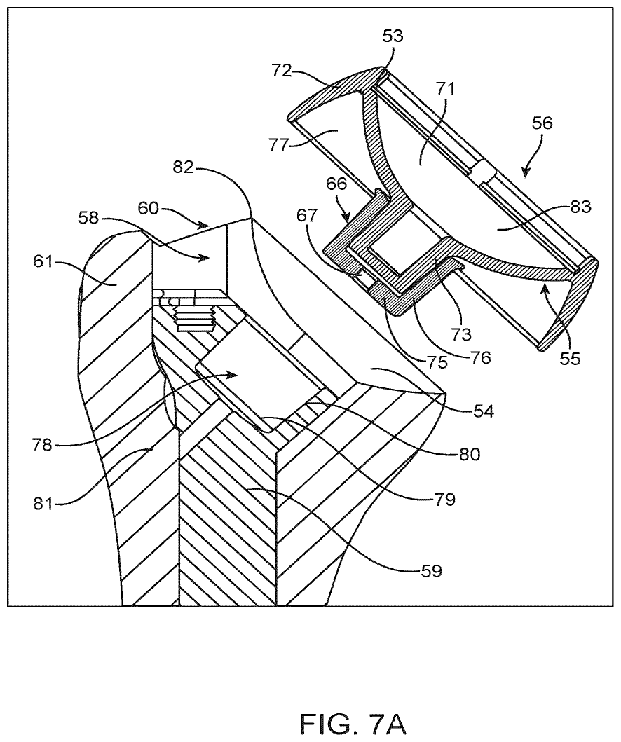

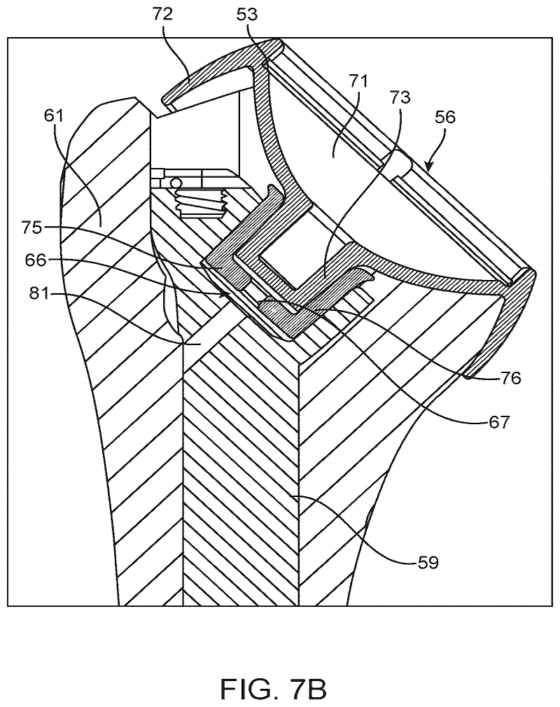

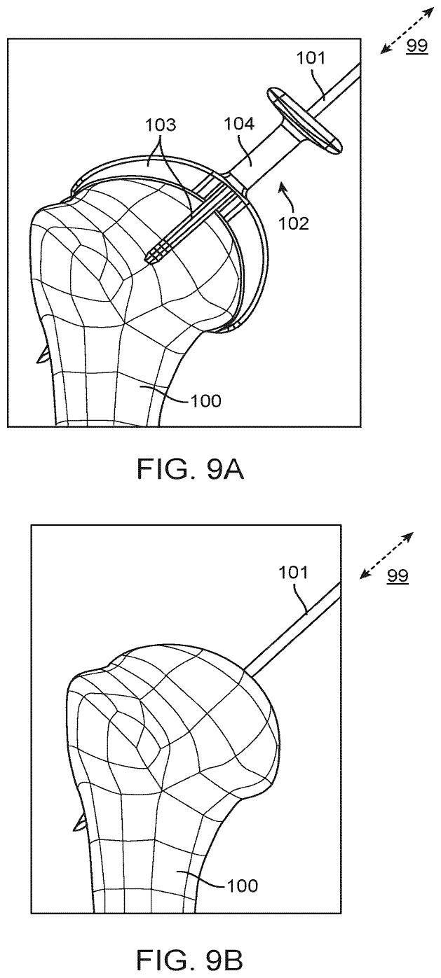

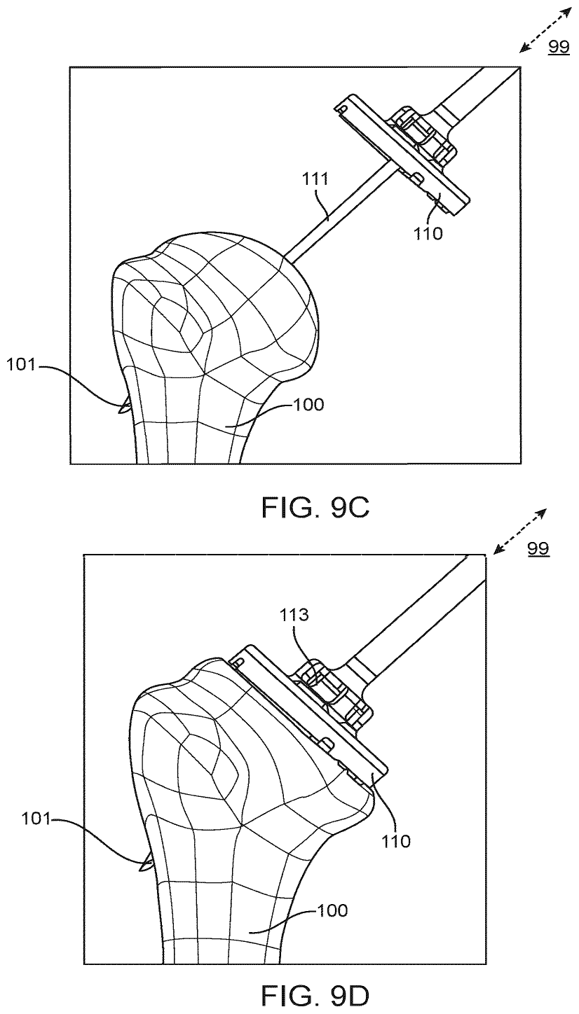

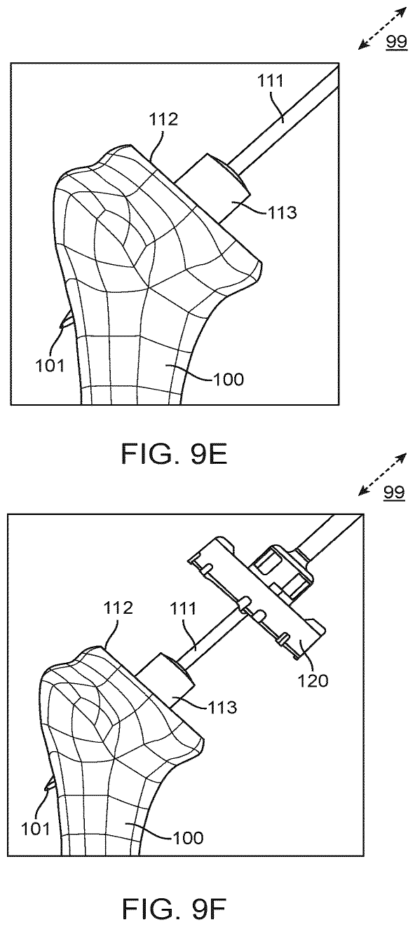

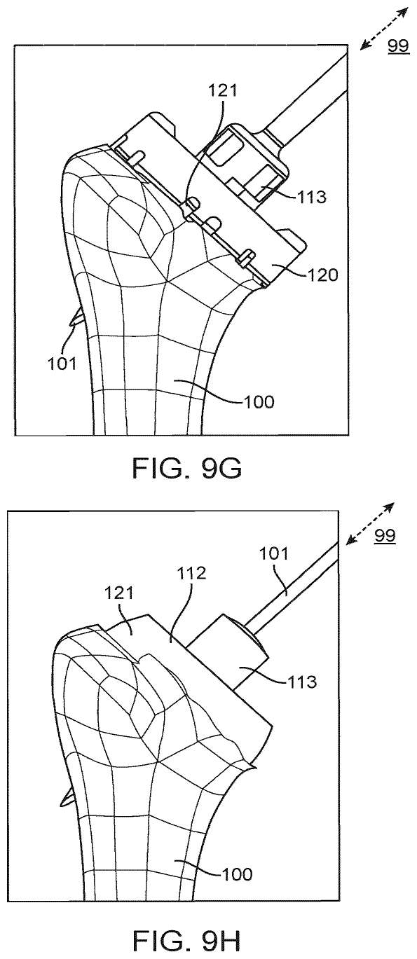

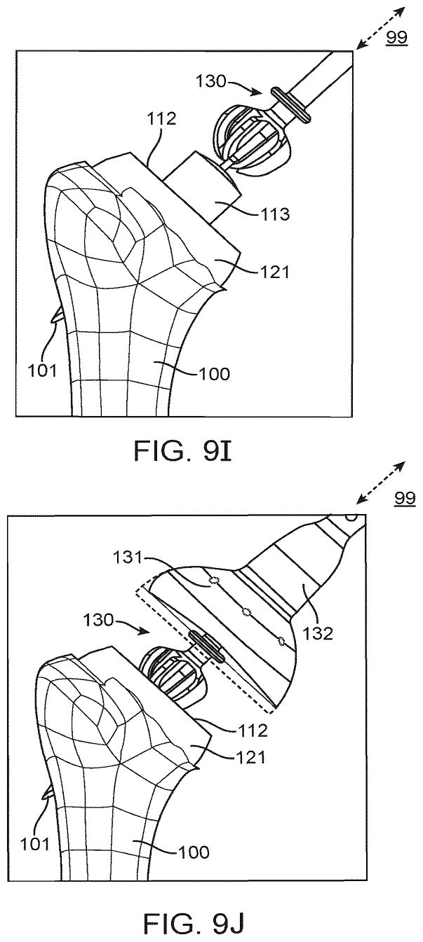

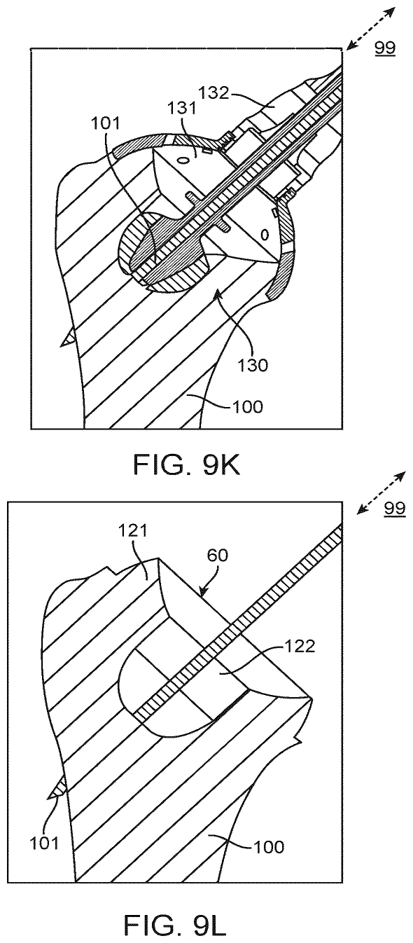

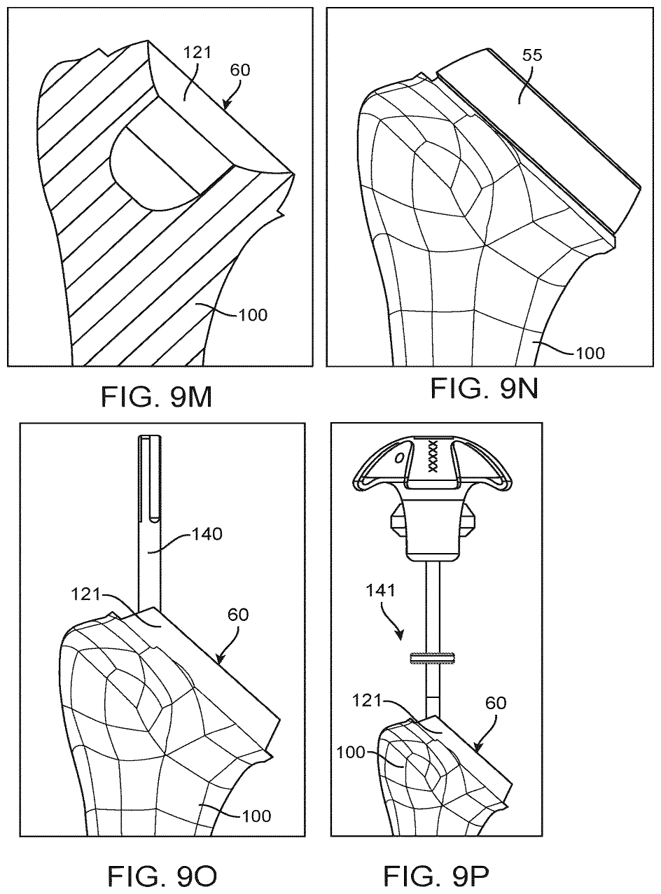

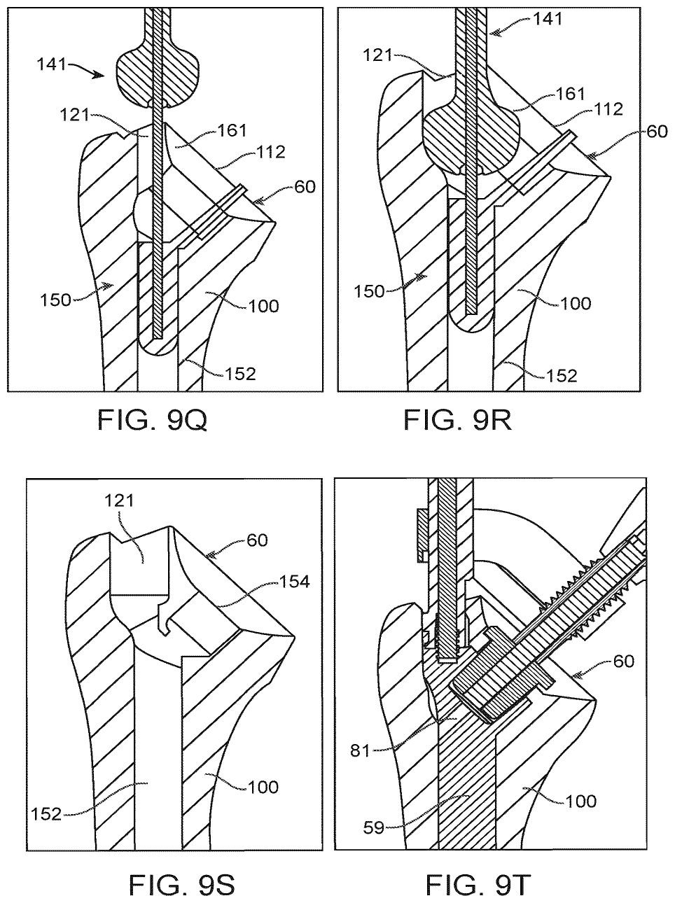

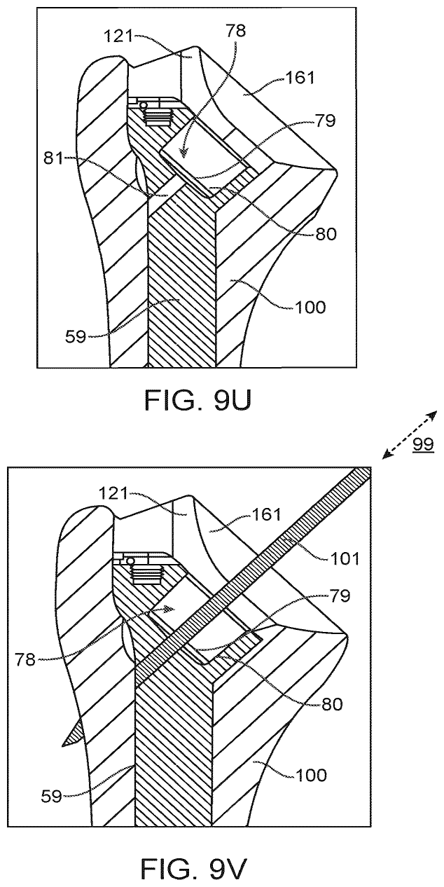

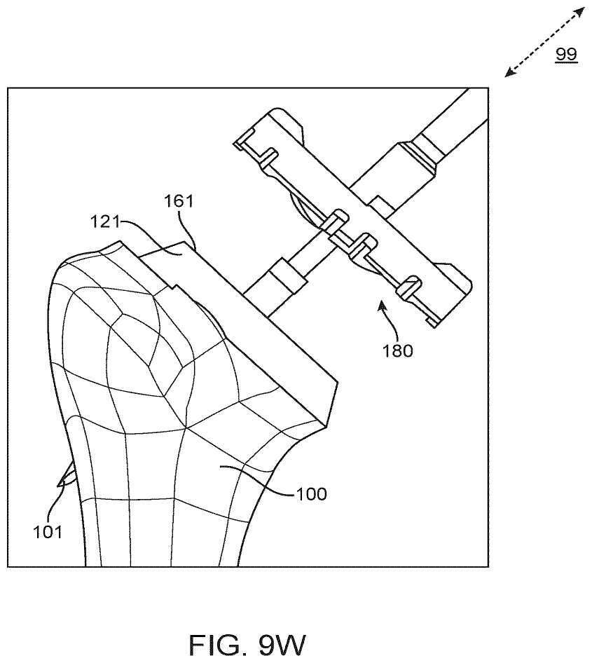

FIGS. 7A and 7B depict at least a portion of an exemplary humeral implant. In some embodiments, the humeral implant is configured to be coupled to the humerus at an excision portion. As used herein, the terms “excision portion” may be used interchangeably with “implant site” or “excision site”. In some embodiments, as described herein, the humeral implant site is located at proximal portion of the humerus (e.g., see FIGS. 9A-9H herein). In some embodiments, the excision portion includes a concave shaped inner implant site portion (e.g., see reference character 54), and an outer implant site surface (e.g., see reference character 58). In some embodiments, the outer implant site surface resembles a ring shape and encircles the inner concave portion. In some embodiments, the inner concave portion defines an implant site opening (e.g., at a bottom portion of the inner implant site portion) providing access to a humeral stem portion (as described herein). As described herein, the bottom portion of the inner concave portion of the implant site may refer to a distal portion of the inner concave portion of the implant site interchangeably.

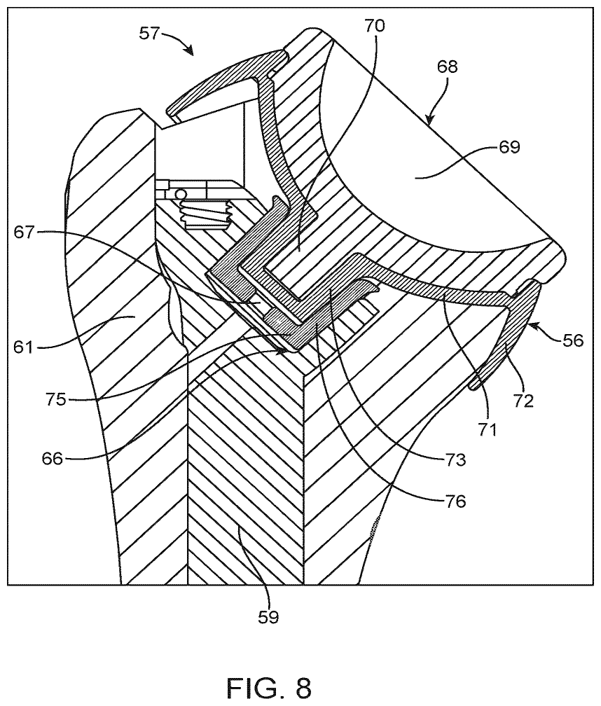

In some embodiments, the humeral implant comprises an implant tray 56 comprising a tray body portion 71, a tray lip portion 72, and a tray base portion 73. A tray body portion 71 may have a concave inner surface 83 oriented to face a liner 68 (see FIG. 8) and a convex outer surface 55 oriented to face the bone at the implant site 60 with a wall therebetween. A tray lip portion 72 of an implant tray 56 is coupled to a proximal portion of the tray body portion 71 of the implant tray 56. In some embodiments, a tray lip portion 72 extends away from the proximal portion of the tray body portion in any direction. In some embodiments, a tray lip portion 72 may extend in a distal direction such that it extends at least partially beyond a tray body portion 71. In some embodiments, a tray lip portion 72 may extend at least partially beyond a tray base portion 73. In some embodiments, a tray lip portion 72 may be oriented perpendicular relative to a proximal end of a tray body portion 71. In some embodiments, a tray lip portion 72 may have an outer convex surface and an inner concave surface 77 and be configured such that the inner concave surface faces a tray body portion 71. In some embodiments, a tray lip portion 72 is a separate component and configured to couple with a tray body portion 71. In some embodiments, the tray lip portion 72 and the tray body portion 71 are a unitary structure.

In some embodiments, a tray base portion 73 of an implant tray 56 extends from a distal portion of the tray body. In some embodiments, the tray base portion extend from the outer convex surface of a tray body portion 71.

In some embodiments, a tray base portion 73 has an opening generally cylindrical in shape. In some embodiments, a tray base portion 73 has an opening in the distal end of the tray body, which defines a tray channel that extend from said tray opening along the longitudinal axis of the tray body portion 71. In some embodiments, a tray body portion 71 opens into the tray channel disposed into a tray base portion 73. In some embodiments, a tray base portion 73 is connected to a tray body portion 71 such that the center of the tray base portion 73 is aligned coaxially with the center of the tray body portion 71. In some embodiments, a tray base portion 73 diameter is smaller than a tray body portion 71 diameter. In some embodiments, a tray base portion 73 is generally cylindrical in shape. In some embodiments, a tray base portion 73 is generally oval, square, rectangular, pentagonal, hexagonal, heptagonal, octanol, nonagonal, or combinations thereof, in shape. In some embodiments, a tray body portion 71, a tray lip portion 72, and a tray base portion 73 are all one piece. In some embodiments, a tray base portion 73 is a separable component and configured to couple with a tray body portion 71.

In some embodiments, an implant tray 56 is configured to be secured to an implant site 60. In some embodiments, the implant tray 56 is configured to be detachably secured to the implant site 60. In some embodiments, the tray lip portion 72 is configured to engage with the outer surface (e.g., 58) of an implant site 60, while the tray body portion 71 is configured to be received by the concave inner portion (e.g., 54) of an implant site 60. In some embodiments, the inner surface of the tray lip portion 72 and the tray body outer surface are disposed and abut about an edge 82 between the outer surface 58 of an implant site 60 and inner surface 54 of an implant site 60. Accordingly, such engagement between i) the inner surface of the tray lip portion 72 and outer surface 58 of the implant site, ii) said interface and edge between the outer surface 58 of implant site 60 and inner surface 54 of an implant site 60, and/or iii) the outer surface of the tray body portion 71 with the inner surface 54 of the implant site 60 secures the implant tray 56 to the implant site 60. In some cases, the implant tray 56 is secured via friction fit between the inner surface of the tray lip and the outer surface 58 of the implant site.

In some embodiments, a tray lip portion 72 is configured to fit around a perimeter wall of the outer surface 58 of an implant site 60. In some embodiments, a tray lip portion 72 of an implant tray 56 may be configured to fit securely around the outside perimeter wall of the outer surface of the implant 60 such that pressure, friction, or a combination of pressure and friction is applied radially inwards by the implant tray 56 to the implant site 60. In some embodiments, pressure, friction, or combination of pressure and friction exerted by an implant tray 56 towards the outer surface 58 of an implant site 60 provides a resistance to decoupling the implant tray 56 and the implant site 60.

Accordingly, in some embodiments, the outer surface 58 includes a tapered or angled profile with reference to an axis orthogonal to the direction at which the tray is inserted into the humeral head (e.g., orthogonal to an axis parallel with the stem bore 81 shown in FIG. 7A). In some embodiments, the contour of the excision site of the humerus at the humeral head, including the outer surface 58 is angled and/or tapered inwards, along a proximal direction, towards a center of the humeral head. In some embodiments, the edge 82 between the outer surface 58 and inner surface 54 forms an angle therebetween, such that in some cases, said edge does not have or does not substantially have a flat (e.g., planar) profile.

In some embodiments, said contour of the excision site, including said edge 82, differs from an excision site for a traditional reverse shoulder replacement procedure, where said excision site may include, for example, an edge between an outer surface and inner surface (similar to 58 and 54 respectively) that has a more flat profile than an embodiment herein. As described herein, the profile of the edge 82 according an embodiment herein helps enable the implant tray to be secured thereto. By contrast, traditional reverse shoulder procedures often do not have implant trays configured to be secured to the excision site (of the humerus) on its own, and therefore, use a more flat profile of the edge at the excision site for the humeral implant. Accordingly, traditional reverse shoulder procedures may require more removal of bone at the excision site, as compared, for example, to an excision site described herein, in order to obtain such a flat profile. In some embodiments, such additional removal of the humeral head, and flat profile is due to the fact that the humeral implant for said traditional reverse shoulder procedure requires a stem to be secured to the humerus bone, so as to secure the humeral implant. By contrast, in an embodiment described herein, since the implant tray 56 is configured to be secured to the outer surface 58 of the humerus excision site (as described herein), a stem is not required for securing the humeral implant thereto (e.g., the stem may just be free floating in the humerus or the stem may not be present at all). Moreover, in such embodiments, the excision can conserve more of the humerus due to the reduced removal amount of the humeral head (as compared to traditional reverse shoulder procedures).

In some embodiments, the thickness between the concave inner surface 83 of the tray body portion 71 and the convex outer surface 55 of the tray body portion 71 is substantially uniform. In some embodiments, at least a portion of the thickness between the inner surface 83 and the outer surface 55 of the tray body portion 71 is variable with at least a first portion having a greater thickness than a second portion. For example, an implant tray 56 with a variable thickness may be selected from a kit of multiple implant trays 56 to account for loss of tissue and/or to prevent the implant tray 56 from falling out of the joint. In some embodiments, one or more fins extend from the outer surface 55 so as to help prevent or reduce an ability of rotation of the implant tray 56 when placed within the excision site. In some embodiments, one, two, three or more fins extend from the outer surface 55 each configured to reduce rotation of the implant tray 56 relative to an implant tray 56 with no fins.

As shown in FIG. 8, a humeral implant 57 may further comprise a liner 68. In some embodiments, a liner 68 may comprise a liner body portion 69 and a liner trunk portion 70. In some embodiments, a liner 68 may be configured to detachably mate with an implant tray 56. In some embodiments, a liner 68 may be configured to fixedly mate with an implant tray 56. In some embodiments, a liner body portion 69 may have a concave inner surface oriented to face the head implant, a convex outer surface oriented to face the implant tray 56, and a wall therebetween. In some embodiments, a convex outer surface of a liner body portion 69 is configured to be received by and mate with a concave inner surface of an implant tray 56. In some embodiments, a liner trunk portion 70 extends from the outer convex surface of a liner body portion 69, such as at the distal end of the liner. In some embodiments, a liner body portion 69 is connected to a liner trunk portion 70 such that a center of the liner trunk portion 70 aligns with the center of the liner body portion 69. In some embodiments, a liner trunk portion 70 diameter is smaller than a liner body portion 69. In some embodiments, a liner trunk portion 70 is generally cylindrical in shape. In some embodiments, a liner trunk portion 70 is generally oval, square, rectangular, pentagonal, hexagonal, heptagonal, octanol, nonagonal, or combinations thereof, in shape. In some embodiments, a liner trunk portion 70 is configured pass through the opening of the tray body and be at least partially received within the channel of a tray base portion 73 (as described herein). In some embodiments, a liner 68 and an implant tray 56 are formed as a unitary component.

In some embodiments, the tray lip further forms a ledge 53 (FIG. 7A) with a proximal portion of the inner tray body surface, which is configured to mate with a surface of the liner. In some embodiments, as described herein, the tray is configured to be secured to the implant site via the tray lip. Accordingly, in some cases, the liner is also secured to the implant site via engagement with the tray. In some embodiments, the liner inner concave surface is configured to receive a head implant. In some embodiments, the head implant is configured to be articulated within the liner, thereby at least partially mimicking a shoulder joint. In some embodiments, the head implant comprises a glenosphere. In some embodiments, as described herein, the glenosphere is coupled to a glenoid portion of the subject.

As shown in exemplary embodiments illustrated in FIGS. 7A, 7B, and 8, a humeral implant 57 further comprises a stem 59 and an adaptor 66. In some embodiments, a stem 59 has a stem cavity 78 disposed in the humeral implant facing surface. In some embodiments, the stem cavity 78 opens towards an opening in the inner surface 54 of the implant site. In some embodiments, the stem cavity 78 is defined by a stem cavity inner wall 80 and a stem cavity bottom surface 79. In some embodiments, a stem bore 81 passes coaxially through a center of the bottom surface 79 and through the body of the stem 59. In some embodiments, a stem bore 81 has an inner diameter larger than the outer diameter of a surgical guide pin. In some embodiments, a stem bore 81 aligns coaxially with an adaptor channel 67 (as described herein) permitting a substantially straight guide pin to pass through both an adaptor channel 67 and the stem bore 81.

In some embodiments, an adaptor 66 may be used to ensure a humeral implant 57 sits flush with the humeral implant site 60. It may be important for the humeral implant 57 to fit flush with the implant site 60 to provide a full range of motion of a shoulder when the humeral implant interfaces with a glenoid implant. The shape of the tray may allow the humeral implant 57 to interface with a minimal amount of bone at the implant site 60 while maintaining sufficient tension to remain secured to the implant site 60.

In some embodiments, an adaptor 66 may have an adaptor foundation (e.g., a base portion of the adapter) 75 and an adaptor wall 76 extending from the adaptor foundation 75. In some embodiments, the inner surface of the adapter foundation 75 and the inner surface of the adapter wall 76 define an adapter channel (e.g., cavity) therein. In some embodiments, an adaptor foundation 75 is generally circular in shape. In some embodiments, an adaptor wall 76 extends from the circumferential edge of an adaptor foundation 75 so that an adaptor 66 forms a cup-like shape. In some embodiments, an adaptor 66 may be generally cylindrical in shape. In some embodiments, an adaptor wall 76 or an outer surface of the adaptor wall 76 is at least partially tapered towards the adaptor foundation 75. In some embodiments, an adaptor wall 76 extends circumferentially from an adaptor foundation 75 at a 90-degree angle. In some embodiments, an adaptor wall 76 extends circumferentially from an adaptor foundation 75 at an angle between 5 to 18 degrees. In some embodiments, an adaptor wall 76 extends circumferentially from an adaptor foundation 75 at an angle between 5 to 18 degrees.

In some embodiments, an adaptor wall 76 extends circumferentially from an adaptor foundation 75 at an angle configured to form a trunnion portion of a Morse taper, where the bore portion is the stem cavity 78. In some embodiments, a Morse taper is formed between the inner surface of the adaptor wall 76 configured as the bore portion of the Morse taper and the tray base portion configured as the trunnion portion of the Morse taper. In some embodiments, a taper or bevel is disposed at least partially at a rim end of an adaptor wall 76 furthest from an adaptor foundation 75. In some embodiments, a bevel or taper disposed at the rim end of an adaptor 66 forms a handle which may be used to remove the adaptor 66 in a revision or subsequent surgery.

In some embodiments, a user may select an adaptor 66 from a kit containing several adaptors with different adaptor foundation 75 thicknesses. In some embodiments, an adaptor 66 has an adaptor foundation 75 thickness of up to 1 mm, 2 mm, 10 mm, or 12 mm. In some embodiments, a user may select an adaptor 66 from a kit containing several adaptors made from different materials. In some embodiments, a user may select an adaptor 66 from a kit containing several adaptors made from different materials, different adaptor foundation 75 thicknesses, different lengths of the adapter, different transverse dimension (e.g., width) of the adapter, or any combination thereof. In some embodiments, an offset measurement is used to select an adaptor 66. In some embodiments, an offset measurement is based on a distance between an opening in the inner surface 54 of the implant site and a bottom surface 79 of the stem cavity. In some embodiments, the offset measurement corresponds to a desired size of the adapter 66 that reduces or eliminates unfilled spacing between the stem cavity 78 inner surface and a tray base portion 73 (e.g., makes a stacked arrangement of the tray 56 (e.g., tray base portion 73), the adapter 66, and the stem 59 more flush with each other). In some embodiments, the desired size of the adapter comprises a thickness of the foundation portion 75, a length of the adapter, a width of the adapter, and/or a thickness of the wall 76. In some embodiments, an adaptor is selected from a kit comprising several adaptors with different foundation thicknesses, transverse thicknesses, materials, or combinations thereof.

In some embodiments, an adaptor 66 may be configured to receive and mate with at least a portion of the implant tray 56. In some embodiments, an opening between an adaptor foundation 75 and the inner surface of an adaptor wall 76 is configured to at least partially receive a tray base portion 73.

In some embodiments, an adaptor 66 may have an adaptor opening 67 disposed from a first surface of an adaptor foundation 75 through a second surface of the adaptor foundation 75. In some embodiments, an adaptor opening 67 has a diameter smaller than a diameter of an adaptor channel. In some embodiments, an adaptor opening 67 is configured to allow a guide pin to pass through the longitudinal axis thereof. In some embodiments, an outer diameter of an adaptor 66 is configured to be at least partially disposed in a stem cavity 78. In some embodiments, a stem 59 may have a stem bore 81 extending at least partially through the body of the stem 59.

In some embodiments, any implant tray described herein (e.g., 56 from FIG. 7a) is configured to interface with a bone (e.g., humerus) when implanted into a subject. In some embodiments, the implant tray is configured to be at least partially porous, thereby helping facilitate bone ingrowth into the pores of the implant tray. Accordingly, in some cases, such bone ingrowth may help integration of the implant tray within the humerus. In some embodiments, at least a portion of the tray body portion 71, the tray lip portion 72, and/or the tray base portion 73 is configured to be porous.

In some embodiments, any stem described herein (e.g., 59 from FIG. 7a) is configured to interface with a bone (e.g., humerus) when implanted into a subject. In some embodiments, the stem is configured to be at least partially porous, thereby helping facilitate bone ingrowth into the pores of the stem. Accordingly, in some cases, such bone ingrowth may help integration of the stem within the humerus.

In some embodiments, the pores for the implant tray and/or the stem are formed using a porous coating. In some embodiments, the implant tray and/or stem have a coating disposed at least partially on the outer surface to promote osseointegration or bone growth at the implant/bone interface or into the implant. For example, the coating may include at least one of tricalcium phosphate, hydroxyapatite, calcium sulfate, calcium carbonate, silver, nitric oxide, antibiotics, antiseptics and antimicrobial peptides with anti-microbial properties, bioceramics, beta-tricalcium phosphate (β-TCP), extracellular matrix proteins, collagen, Arg-Gly-Asp (RGD) peptides, biological peptides, fibroblast growth factor 2 (FGF2), transforming growth factor-β (TGF-β) including TGF-β2, bone morphogenic proteins (BMP) including BMP2 and BMP7, chitosan, any growth factors known to those skilled in the art to impart bioactivity and biocompatibility to the surface of an implant so as to promote bone ingrowth and differentiation of native cells into desirable cell lineages such as osteoblasts leading to enhanced osteointegration of the implant, or any combination thereof. In some embodiments, a porous plasma spray is applied to the outer surface of the implant tray and/or stem to create and porous outer surface. For example, the porous plasma spray may create a layer on the outer surface of the implant tray and/or stem such that biologic fixation is improved relative to an implant with no porous plasma spray. In some embodiments, the coating is adapted to provide additional resistance to shear and/or axial forces acting upon the implant.

In some embodiments, for any stem described herein (e.g., 59 from FIG. 7A), at least a portion of the stem comprises a bioresorbable material. Accordingly, in some embodiments, the at least a portion of the stem comprising a bioresorbable material is configured to be absorbed within the humerus bone over time. For example, the degradation of the bioabsorbable material may be selected such that it degrades at the same or a substantially similar rate as new bone grows. In some embodiments, the bioresorbable material comprises at least one of poly(glycolic acid), poly(lactic acid), poly(lactic-co-glycolic acid), poly(glycolide-co-trimethylene carbonate, collagen, cellulose, chitosan, silk, demineralized bone matrix, bioceramics, magnesium alloys, poly(caprolactone), poly (p-dioxanone), poly(ortho esters), poly(aryletherketones), poly(ether ether ketone), hyaluronic acid, derivatives thereof, any copolymer thereof, and any combination thereof. A resorbable stem may provide numerous advantages over traditional stems. For example, in a revision surgery it is less invasive to place a new stem in bone then to remove a stem from bone and place a new bone. Likewise, for younger patients who outgrow their original implant, having an implant that is capable of being fully resorbed may mean that they would not need a revision surgery as they get older.

### Offset Tool

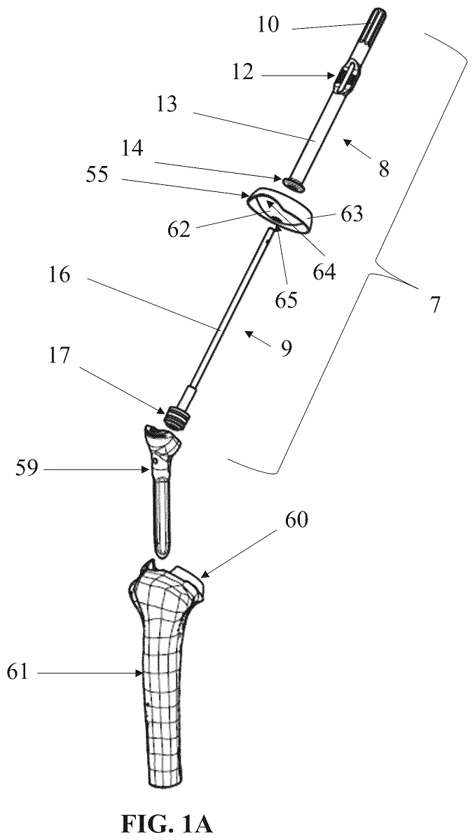

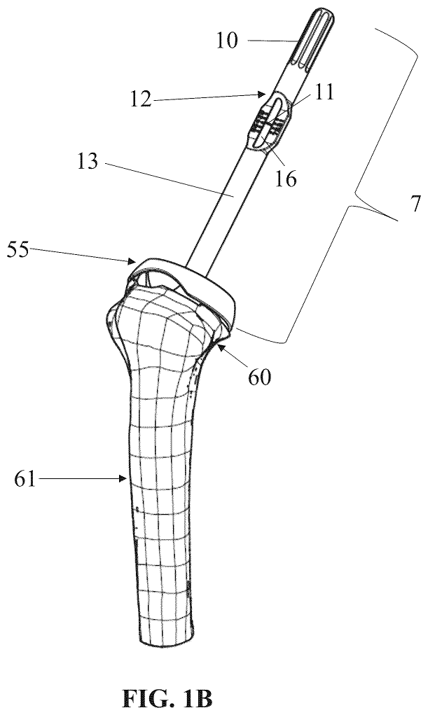

In some embodiments, a humeral implant placement system includes an offset tool for determining a desired size of an adapter, so as to make the coupling between the tray and the stem more flush (e.g., reduces or eliminates unfilled spacing between the tray (e.g., base portion) and the stem cavity inner surface when stacked together). Depicted in FIG. 1A is an exemplary offset tool 7 having an offset tool proximal portion 8 and an offset tool distal portion 9 as shown in FIG. 1A. In some embodiments, an offset tool proximal portion 8 comprises an offset measuring tool handle 10, an offset measuring tool scale display 12, an offset tool outer shaft 13, and an offset tool tray interfacing member 14. The offset measuring tool handle 10 may be configured to be held by a user when using the offset tool 7. The offset tool proximal portion 8 may have an opening at least partially disposed within an offset tool outer shaft 13. In some embodiments, the offset tool outer shaft 13 defines a channel therein extending longitudinally (with respect to the offset tool outer shaft) from the opening in the offset tool outer shaft and is configured to allow an offset tool inner shaft 16 to pass through said opening. In some embodiments, an offset tool 7 has an offset tool distal portion 9 having an offset tool inner shaft 16 and an offset tool stem interfacing member 17. In some embodiments, an offset tool stem interfacing member 17 is configured to fit in a stem cavity 78. In some embodiments, an offset tool 7 is configured to mate with a trial tray 55.

FIG. 1B illustrates an exemplary compact configuration of an offset tool 7. In some embodiments, an offset tool inner shaft 16 is disposed within an offset tool outer shaft 13. An offset tool inner shaft 16 may have an offset marking bar 11. In some embodiments, when an offset tool 7 is in a compact form, an offset marking bar 11 is visible through an opening in an offset measuring tool scale display 12. In compact form, an offset tool 7 may be configured so an offset tool tray interfacing member 14 abuts an external facing surface of a trial tray 55 while an offset tool inner shaft 16 passes through a trial tray open passage 65 of said trial tray 55, and an offset tool stem interfacing member 17 is at least partially received in a stem 59 via a stem opening (not shown), wherein the stem is at least partially disposed within a bone 61. A trial tray 55 may be configured to mate with an implant site 60 located on the humerus bone.

In some embodiments, an offset marking bar 11 is a groove disposed at least partially within an offset tool inner shaft 16 and visible from an exterior of the inner shaft. In some embodiments, an offset marking bar 11 is disposed on an offset tool inner shaft 16 (e.g., on an exterior surface of the offset). In some embodiments, an offset marking bar 11 may be a line oriented perpendicular to a longitudinal axis of an offset tool inner shaft 16. An offset marking bar 11 may be partially disposed around the circumference of an offset tool inner shaft 16. An offset marking bar 11 may be entirely disposed around the circumference of an offset tool inner shaft 16.

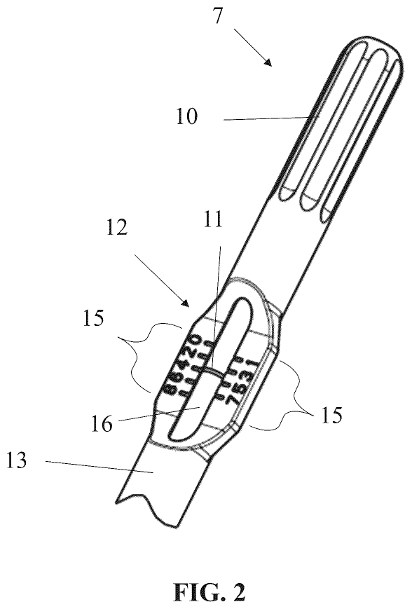

FIG. 2 illustrates an embodiment of an offset tool 7 having an offset measuring tool scale display 12 connected to an offset tool outer shaft 13. In some embodiments, an offset measuring tool scale display 12 is disposed between an offset measuring tool handle 10 and an offset tool stem interfacing member 17. In some embodiments, the offset measuring tool scale display 12 is configured to display to the user the offset (e.g., distance) between a stem 59 and a trial tray 55 (e.g., trial tray location about an opening (e.g., see 122 in FIG. 9L) in the concave inner surface of the implant site 54). In some embodiments, offset measuring tool scale display 12 is configured to display to the user the offset between a stem 59 and an implant tray 56 (as described herein, e.g., FIG. 7A). In some embodiments, an offset measuring tool scale display 12 may include offset tool scale markings 15. In some embodiments, offset tool scale markings 15 has tick marks or lines, and numbers corresponding to one or more tick mark. In some embodiments, an offset tool scale markings 15 are tick marks or lines oriented substantially perpendicular to the longitudinal axis of an offset tool 7. In some embodiments, offset tool scale markings 15 are tick marks showing the offset (e.g., distance) between an offset tool tray interfacing member 14 and an offset tool stem interfacing member 17. In some embodiments, the offset tool scale markings 15 are tick marks or lines with corresponding numbers used to determine an offset between a stem 59 (or portion thereof) and a trial tray 55. In some embodiments, the offset tool scale markings 15 are tick marks or lines with corresponding numbers used to determine an offset between a stem 59 and an implant tray 56. In some embodiments, the offset tool scale markings 15 lines or numbers correspond to an offset or gap distance according to any unit of measure for length, such as millimeters, centimeters, inches, etc. In some embodiments, the numbers itself represent the unit of measure. In some embodiments, the numbers represent a scale (e.g., 1 to 10), wherein each number on the scale corresponds to a distance according to a unit of measure. In some embodiments, the offset tool markings 15 correspond to the selection of an adaptor 66 from a plurality of adaptor sizes.

In some embodiments, an offset measuring tool scale display 12 may have a window or opening disposed through an offset tool outer shaft 13. In some embodiments, an opening in an offset measuring tool scale display 12 extends along the longitudinal axis of an offset tool outer shaft 13. In some embodiments, the opening in an offset measuring tool scale display 12 may have an offset tool scale markings 15 disposed on at least one side of the opening. In some embodiments, an offset tool inner shaft 16 can be seen through the opening in an offset measuring tool scale display 12. In some embodiments, an offset marking bar 11 can be seen through the opening in an offset measuring tool scale display 12. In some embodiments, an offset marking bar 11 aligns with a line of an offset tool scale markings 15. In some embodiments, when an offset marking bar 11 aligns with a line of the offset tool scale markings 15, a number corresponding to the line indicates the offset (according to the respective unit of measure, such as millimeters).

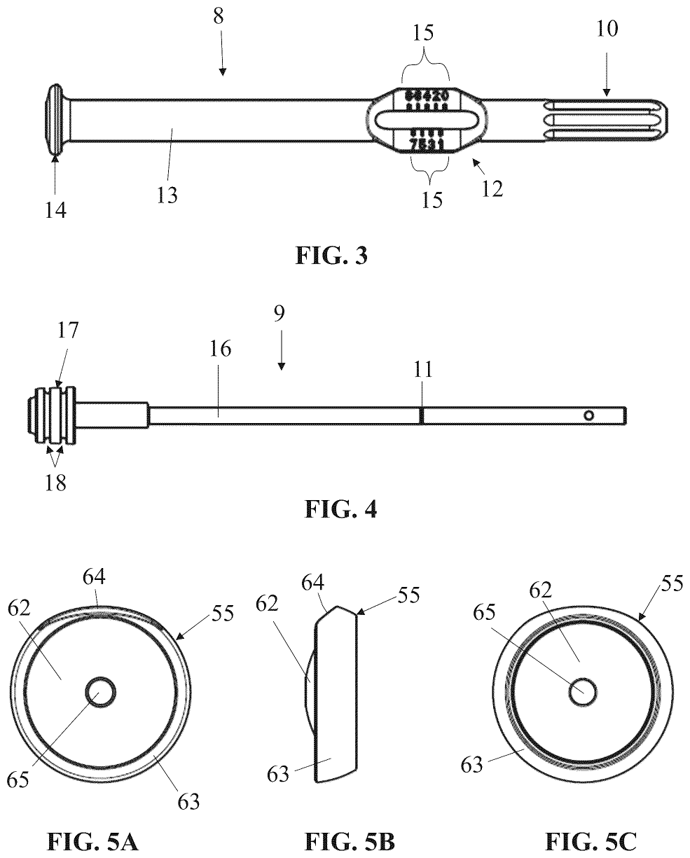

FIGS. 3 and 4 illustrate an embodiment of an offset tool proximal portion 8 and an offset tool distal portion 9. In some embodiments, an offset tool proximal portion 8 has an elongated substantially cylindrical shape. An offset tool proximal portion 8 may have an offset measuring tool handle 10 disposed at one end and an offset tool tray interfacing member 14 disposed at the other end. An offset measuring tool handle 10 may have at least one groove recessed therein and extending along the longitudinal axis of an offset measuring tool handle 10. An offset tool proximal portion 8 may have an offset tool outer shaft 13 disposed between an offset measuring tool handle 10 and an offset tool tray interfacing member 14. An offset tool outer shaft 13 may have an opening at least partially extending through an offset tool outer shaft 13 along the longitudinal axis therein. In some embodiments, an offset tool outer shaft 13 may have an offset measuring tool scale display 12 at least partially extending from the outer surface of an offset tool outer shaft 13. In some embodiments, an offset tool outer shaft 13 may have an offset measuring tool scale display 12 at least partially recessed in the outer surface of an offset tool outer shaft 13.

In some embodiments, an offset tool tray interfacing member 14 is configured to have a diameter larger than an outer diameter of an offset tool outer shaft 13. In some embodiments, a distal end of an offset tool tray interfacing member 14 has a convex outer surface. In some embodiments, a convex outer surface of the distal end of an offset tool tray interfacing member 14 is configured with a curvature to mate with the external facing surface of a trial tray 55.

An offset tool distal portion 9 may have an offset tool inner shaft 16 extending from a first end to a second end. The outer diameter of an offset tool inner shaft 16 may be smaller than the channel extending within and through the offset tool outer shaft 13. Accordingly, in some embodiments, the offset tool inner shaft is configured to be received within the channel of the outer shaft. In some embodiments, the inner shaft is configured to move in a telescopically manner through the channel of the outer shaft (as described herein). In some embodiments, the diameter of an offset tool inner shaft 16 is smaller than the outer diameter of an offset tool outer shaft 13. An offset tool distal portion 9 may have an offset tool stem interfacing member 17 disposed at the first or second end. An offset tool stem interfacing member 17 may have a diameter greater than the diameter of an offset tool inner shaft 16. In some embodiments, an offset tool stem interfacing member 17 has one or more offset tool grooves 18 recessed at least partially through an outer surface of an offset tool stem interfacing member 17 and extending around the circumference thereof.

In some embodiments, the grooves 18 are configured to removably engage with a stem cavity 78. In some embodiments, an offset tool stem interfacing member 17 may have at least one offset tool grooves 18 configured to fit an O-ring. In some embodiments, an offset tool stem interfacing member 17 has two offset tool grooves 18 wherein each groove is configured to fit an O-ring. An offset tool groove 18 may be configured such that when an O-ring is placed in the groove, it forms a seal between an offset tool stem interfacing member 17 and a stem 59. In some embodiments, the grooves 18 are configured to reduce friction between the offset tool stem interfacing member 17 and the stem cavity 78 and facilitate removal of the offset tool stem interfacing member 17 from the stem cavity 78 with minimal to no movement of the stem 59.

In some embodiments, an offset tool stem interfacing member 17 may have a first portion connected to an offset tool inner shaft 16 having a diameter larger than an offset tool inner shaft 16. In some embodiments, an offset tool stem interfacing member 17 has a first portion and a second portion connected to the first portion where the second portion has a diameter larger than the first portion. In some embodiments, an offset tool stem interfacing member 17 has a third portion connected to the second portion and having a smaller diameter than the second portion and a larger diameter than the offset tool inner shaft 16. In some embodiments, an offset tool stem interfacing member 17 has a third portion that is tapered away from the second portion. In some embodiments, the stem interfacing member 17 comprises threading on its exterior surface. In some embodiments, such threading is configured to threadably engage with an inner portion of the stem (as described herein).

FIGS. 5A-C illustrate an exemplary embodiment of a trial tray 55 having a trial tray body portion 62, a trial tray lip portion 63, a trial tray lip indentation 64, and/or a trial tray open passage 65. In some embodiments, a trial tray body portion 62 has a convex bone facing surface and a concave external facing surface. In some embodiments, a trial tray lip portion 63 extends from a rim of a trial tray body portion 62 distally. In some embodiments, a trial tray 55 has a trial tray open passage 65 disposed through the center of the surface of the trial tray body portion 62. In some embodiments, a trial tray open passage 65 is configured to permit an offset tool inner shaft 16 to pass through the open passageway. In some embodiments, a diameter of a trial tray open passage 65 is configured to prevent an offset tool tray interfacing member 14 from passing through the open passageway. In some embodiments, a trial tray 55 has a trial tray lip indentation 64 disposed in a trial tray lip portion 63. In some embodiments, a trial tray lip indentation 64 is configured to provide a handle area for a user to remove a trial tray 55 from an implant site 60. In some embodiments, a trial tray 55 is temporarily placed on an implant site 60 prior to attaching an implant tray 56 to the implant site 60.

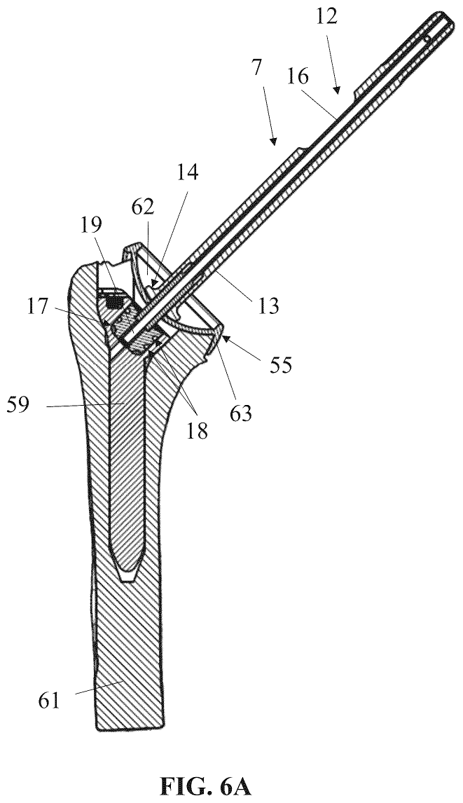

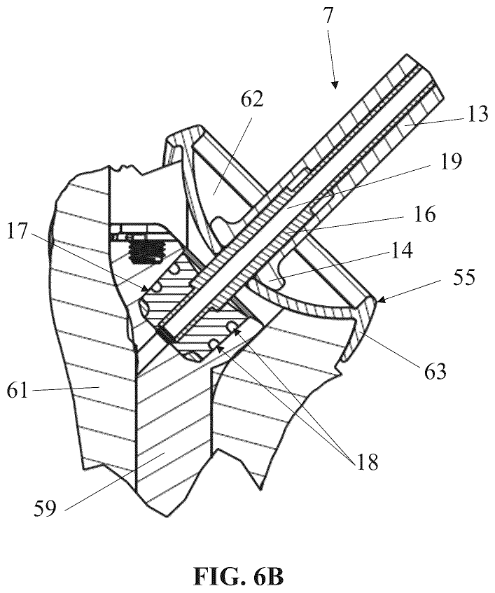

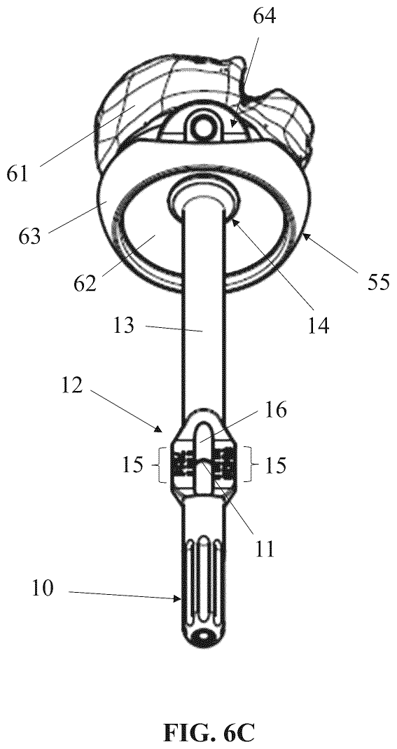

FIGS. 6A-C illustrate an embodiment of an offset tool 7 in a compacted configuration interfacing with a stem 59 disposed within a bone 61. An offset tool stem interfacing member 17 may be configured to fit within a stem cavity 78 (not shown). In some embodiments, of an offset tool stem interfacing member 17 is at least partially tapered narrowing towards the most distal end. In some embodiments, a tapered end of an offset tool stem interfacing member 17 abuts the inner surface of a stem cavity 78. In some embodiments, a distal end of an offset tool stem interfacing member 17 abuts the inner surface of the stem cavity 78.

As shown in an embodiment shown in FIG. 6C, an offset tool 7 in a compact configuration has an offset tool inner shaft 16 which telescopically interfaces with an outer shaft 13, such that the inner shaft is configured to extend distally through a channel within the outer shaft so as to be seen through an opening in an offset measuring tool scale display 12 and an offset tool outer shaft 13. In some embodiments, when an offset tool stem interfacing member 17 interfaces with a stem 59 and an offset tool tray interfacing member 14 interfaces with a trial tray body portion 62, an offset marking bar 11 on an offset tool inner shaft 16 can be seen through an opening in an offset measuring tool scale display 12 and an offset tool outer shaft 13. In some embodiments, an offset marking bar 11 aligns with an individual line within offset tool scale markings 15.

In some embodiments, an offset tool distal portion 9 has an offset tool open channel 19 disposed at least partially through the longitudinal axis. In some embodiments, an offset tool open channel 19 extends from a distal end of an offset tool stem interfacing member 17 along the longitudinal axis of an offset tool distal portion 9 and at least partially through an offset tool inner shaft 16. In some embodiments, an offset tool open channel 19 extends from the distal end an offset tool through at least a portion of an offset tool proximal portion 8 and an offset tool distal portion 9. The offset tool open channel 19 may be configured to permit a surgical guide pin to pass through the offset tool open channel 19. The offset tool open channel 19 may have a diameter greater that the diameter of a surgical guide pin. In some embodiments, an offset tool tray interfacing member 14 abuts the external facing surface of a trial tray body portion 62. In some embodiments, an offset tool inner shaft 16 passes through a trial tray open passage 65 of a trial tray 55.

In some embodiments, offset tool measuring system comprising an offset tool 7, a trial tray 55, and a stem 59 may be used to determine the offset or gap length between a stem 59 and an implant tray 56 (e.g., a distance from a stem bottom surface 79 and tray 55). In some embodiments, an offset tool 7 is configured to pair with a trial tray 55 to measure the offset or gap length.

In some embodiments, a method to determine an offset comprises the steps of placing an offset tool stem interfacing member 17 in a stem cavity 78, pushing the offset tool stem interfacing member 17 to the stem cavity bottom surface 79 the stem cavity 78, sliding a trial tray 55 over an offset tool inner shaft 16 so the offset tool inner shaft 16 is disposed within a trial tray open passage 65, sliding an offset tool outer shaft 13 over an offset tool inner shaft 16 so an offset tool tray interfacing member 14 abuts a trial tray body portion 62, determining the offset tool scale markings 15 individual marking which aligns with an offset marking bar 11.

FIGS. 9A-W illustrate methods, systems, and tools used to prepare and place a humeral implant in a shoulder surgery (e.g., reverse shoulder surgery). In some embodiments, when placing a humeral implant 57 in a patient, a working axis 99 is established that corresponds to a proper angle of insertion (of the humeral implant) into a humerus 100. As shown in FIG. 9A, a working axis 99 may be designated by disposing a surgical guide pin 101 into a humerus 100 along the working axis 99. The surgical guide pin 101 may serve as a landmark to the surgeon for where the working axis 99 is located through the humerus 100. A surgical guide pin 101 may be inserted into a humerus 100 using a pin guide alignment device 102 to ensure the longitudinal axis of the surgical guide pin 101 passes through a working axis 99. In some embodiments, a pin guide alignment device 102 may have one or more pin guide alignment device arms 103 extending radially from the longitudinal axis of a pin guide alignment device body portion 104. In some embodiments, a pin guide alignment device 102 has four pin guide alignment device arms 103. In some embodiments, one or more pin guide alignment device arms 103 are curved towards a humerus 100 epiphysis. In some embodiments, one or more pin guide alignment device arm(s) 103 are configured to mate with the proximal articulating surface of a humerus 100 (e.g., at least a portion of the humeral head). A pin guide alignment device 102 may have a bore (not shown) extending through a longitudinal axis of pin guide alignment device body portion 104. In some embodiments, the bore may be configured to permit a surgical guide pin 101 to pass through the bore therein. In some embodiments, a surgical guide pin 101 passes through a bore disposed in a pin guide alignment device 102 and into a humerus 100 along a working axis 99. In some embodiments, the surgical pin guide is inserted into the humerus 100 using a drill, a hammer, or other device as known in the art.

As shown in FIG. 9B, once a surgical guide pin 101 has been inserted into a humerus 100, a pin guide alignment device 102 may be removed by sliding the pin guide alignment device 102 away from the articulating surface of a humerus 100 along a working axis 99 until it is free from the surgical guide pin 101.

A flat reamer 110 may have an opening disposed through a longitudinal axis (not shown). In some embodiments, the flat reamer 110 is advanced along a working axis 99 towards a humerus 100 such that a surgical guide pin 101 passes through the opening in the flat reamer 110. In some embodiments, such as illustrated in FIG. 9C, the flat reamer 110 is advanced along a working axis 99 towards a humerus 100 such that a centering shaft 111, disposed around a surgical guide pin 101, passes through the opening in the flat reamer 110. In some embodiments, a centering shaft 111 may be at least partially inserted into a humerus 100 and have a longitudinal axis extending along a working axis 99. In some embodiments, the centering shaft may include threads allowing a shaft to be threaded into the humerus.

As illustrated in FIG. 9D, a flat reamer 110 may interface with the articulating surface of a humerus 100 and rotate to ream a humerus 100 to form a flat bone surface 112 (not shown in 9D) and an inner bone cylinder 113. In some embodiments, the inner bone cylinder is formed based on a stopper function (not shown) of the flat reamer, thereby restricting an amount of bone removed from the portion of the humerus. In some embodiments, a flat reamer 110 may be an OVOMotion™ reamer. The flat reamer 110 may be removed from the surgical area by sliding away from a humerus 100 along a working axis 99 and over a centering shaft 111 encircling a surgical guide pin 101. As shown in FIG. 9E after the flat reamer 110 has reamed the articulating surface of a humerus 100, the articulating surface has a flat bone surface 112 of bone disposed generally perpendicular to working axis 99. In some embodiments, the resulting humeral surface after reaming with the flat reamer 110 has an inner bone cylinder 113 extending in a proximal direction away from an inner portion of a flat bone surface 112 in a substantially cylindrical shape. In some embodiments, the longitudinal axis of an inner bone cylinder 113 is aligned with a working axis 99. In some embodiments, an inner bone cylinder 113 may have an outer diameter smaller than a flat bone surface 112 outer diameter.

As shown in FIG. 9F a concave reamer 120 may be passed over a centering shaft 111 along a working axis 99 and towards a flat bone surface 112. As shown in FIG. 9G a concave reamer 120 may rotate and ream at least a portion of an outer surface of a humerus 100 extending distally from a flat bone surface 112. In some embodiments, a concave reamer 120 reams the external surface of a humerus 100 to form a circumferential bone surface 121. In some cases, the circumferential bone surface corresponds to the outer surface of the excision or implant site 60, as described herein (see e.g., ref. char. 58 in FIG. 7A). In some embodiments, the concave reamer 120 further reams an interior surface of the humerus, thereby forming an inner concave surface. In some embodiments, said inner concave surface corresponds to the inner concave surface of the implant site, as described herein (see e.g., ref. char. 54 in FIG. 7A). As illustrated in FIG. 9H, a concave reamer 120 may be removed by sliding it along a working axis 99 away from a flat bone surface 112 and over a centering shaft 111 encircling a surgical guide pin 101. In some embodiments, the concave reamer 120 includes a stopper so as to limit the amount of bone removed from the humerus. In some embodiments, a circumferential bone surface 121 has a convex curved shape. In some embodiments, a centering shaft 111 is removed after a concave reamer 120 has reamed a humerus 100.

As illustrated in FIG. 9I, an angled reamer 130 may be passed over a surgical guide pin 101 towards humerus implant site. As shown in FIG. 9J, an angled reamer 130 may ream a humerus 100 from a proximal end of an inner bone cylinder 113 and distally to an inner surface of the implant site of the humerus (e.g., bounded by a circumferential bone surface 121). In some embodiments, the angled reamer 130 forms an opening 122 at a distal end of the inner concave surface of the implant site. In some embodiments, an angled reamer 130 may ream a humerus 100 at least partially to a spongy bone region of an epiphysis, a medullary cavity, or a combination of spongy bone and medullary cavity of the humerus 100. As illustrated in FIG. 9J, a first handle portion 131 connected to a distal end of a second handle portion 132 may be passed over a surgical guide pin 101 and an angled reamer 130, wherein a surgical guide pin and a shaft for the angled reamer are configured to pass through an opening of the first and second handle portion. A first handle portion 131 may have a concave curved outer surface. As shown in FIG. 9K a first handle portion 131 may be configured to fit over a circumferential bone surface 121. As shown in FIG. 9L, an angled reamer 130, a first handle portion 131, and a second handle portion 132 may be removed from the surgical field by sliding the members in a direction away from a humerus along a working axis 99. In some embodiments, a humerus channel 152 extends from an opening 154 formed in the inner concave surface 161 of the implant site 60 to the medullary cavity.

As illustrated in FIG. 9M, a surgical guide pin 101 may be removed after an angled reamer 130, a first handle portion 131, and a second handle portion 132 have been removed from the surgical field leaving a reamed humerus 100. As shown in FIG. 9N a trial tray 55 may be fit over a circumferential bone surface 121. As shown in FIG. 9O, a trial tray 55 may be removed and a pilot hole may be drilled into the proximal surface of a humerus 100 with a drill 140. The drill 140 may be removed once a pilot hole is disposed in a humerus 100. As shown in FIG. 9P, a stem reamer 141 may be used to ream the pilot hole (not shown) in the humerus 100. As shown in FIGS. 9Q-9T a canal guide 150 can be inserted within a humerus 100 and used to guide a stem reamer 141 into a proximal surface of the humerus 100. In some embodiments, the opening created by a stem reamer 141 is configured to permit a stem 59 to pass therethrough (which may correspond to stem 59 as described herein, for e.g., in FIGS. 7A-8.

As depicted in FIGS. 9U and 9V once a stem 59 has been placed in a humerus 100, a surgical guide pin 101 may be inserted along a working axis 99 through an inner concave surface 161 and a stem 59. In some embodiments, a stem 59 has a stem cavity 78 disposed in an implant facing surface. In some embodiments, a stem cavity 78 is generally cylindrical in shape. In some embodiments, a stem cavity 78 is generally oval, square, rectangular, pentagonal, hexagonal, heptagonal, octanol, nonagonal, or combinations thereof, in shape. In some embodiments, a stem 59 has a stem bore 81 extending through a working axis 99. A stem bore 81 may pass through a longitudinal axis of a stem cavity 78. In some embodiments, a diameter of a stem bore 81 is smaller than a diameter of a stem cavity 78. In some embodiments, a diameter of a stem bore 81 is configured to receive a surgical guide pin 101 therein. In some embodiments, a stem 59 is disposed at least partially within a humerus 100. In some embodiments, a stem 59 is disposed at least partially within a humerus 100 and secured at least partially by coupling with an adaptor 66, an implant tray 56, a liner 68, or combinations thereof, as described herein. In other cases, the stem 59 is further secured, or alternatively secured, at least partially with bone cement and/or threading (e.g., on an outer surface of the stem).

As shown in FIG. 9W, a cleanup reamer 180 may be passed over a surgical guide pin 101 along a working axis 99 towards a curved bone surface 161 of a humerus 100. A cleanup reamer 180 may have a bore extending through the longitudinal axis. A bore disposed in a cleanup reamer 180 may be configured to have a larger diameter than a surgical guide pin 101. A cleanup reamer 180 may be removed from the implant site by sliding the cleanup reamer 180 away from an inner concave surface 161 along a working axis 99 until the cleanup reamer 180 no longer has a surgical guide pin 101 disposed within the cleanup reamer 180 bore. In some embodiments, a cleanup reamer 180 is configured to ream an inner concave surface 161 until the rim of an inner concave surface 161 is substantially perpendicular to a working axis 99. In some embodiments, the cleanup reamer 180 is configured to ream the proximal end of the humerus bone 100 to ensure it is true and properly aligned with the stem 59.

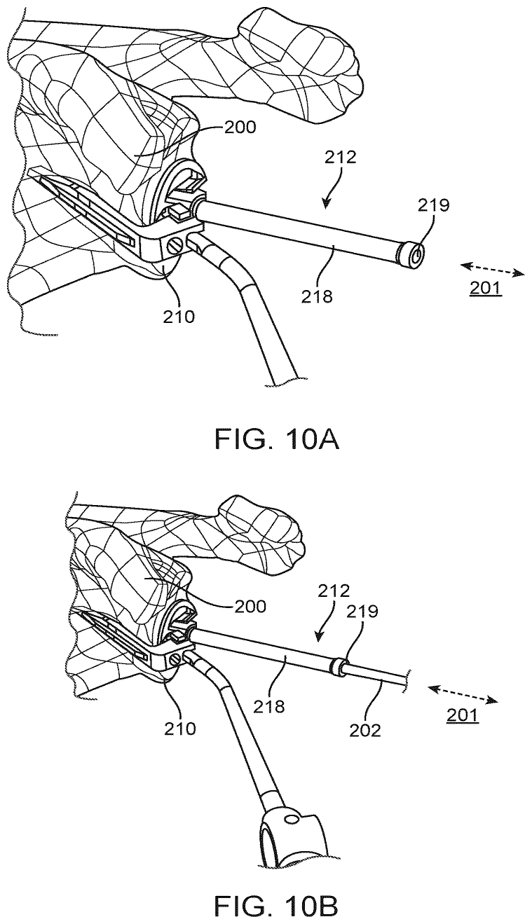

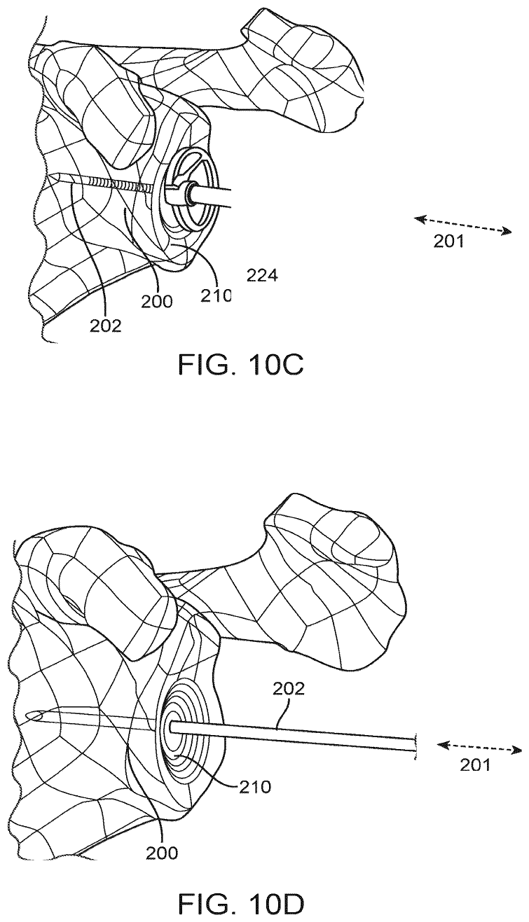

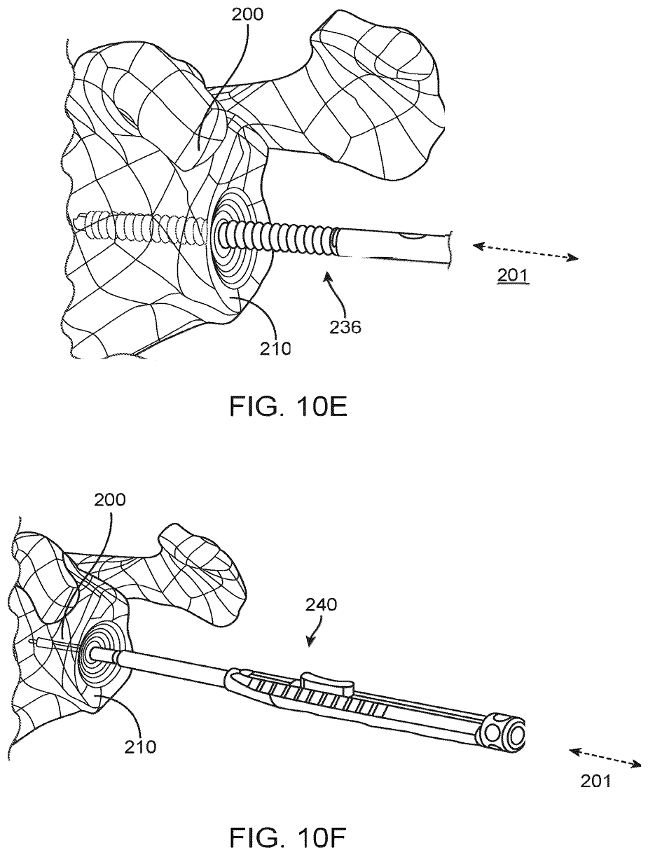

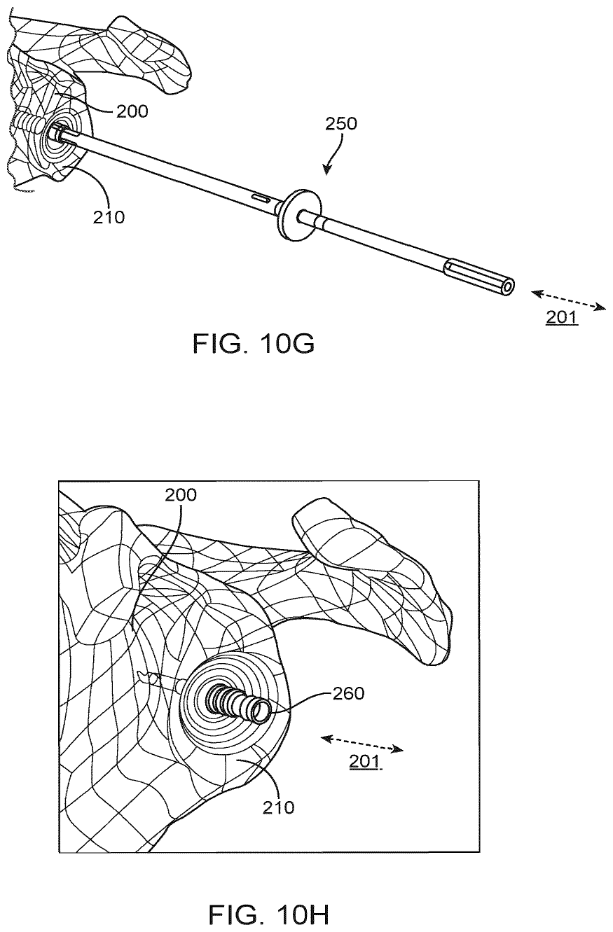

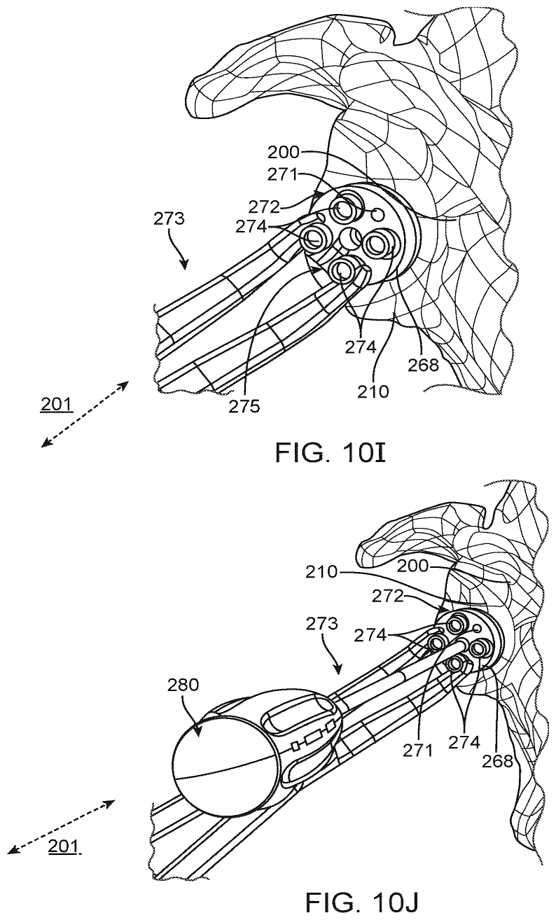

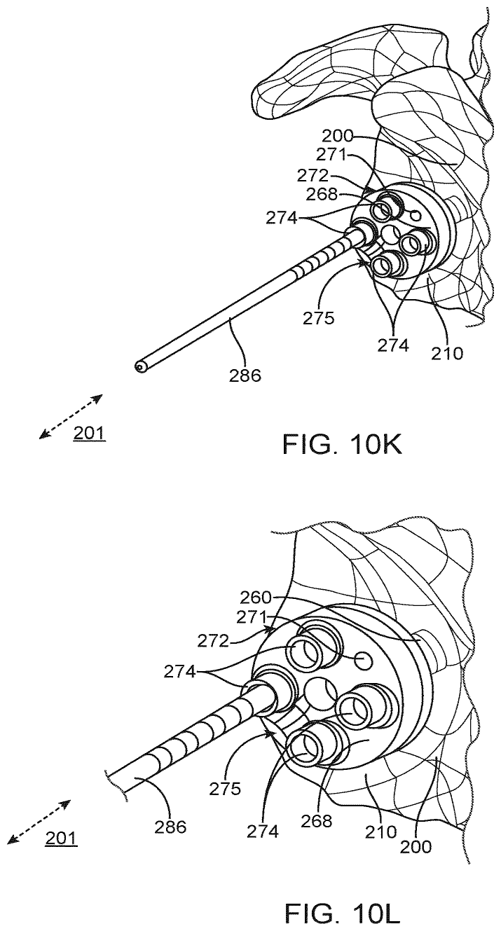

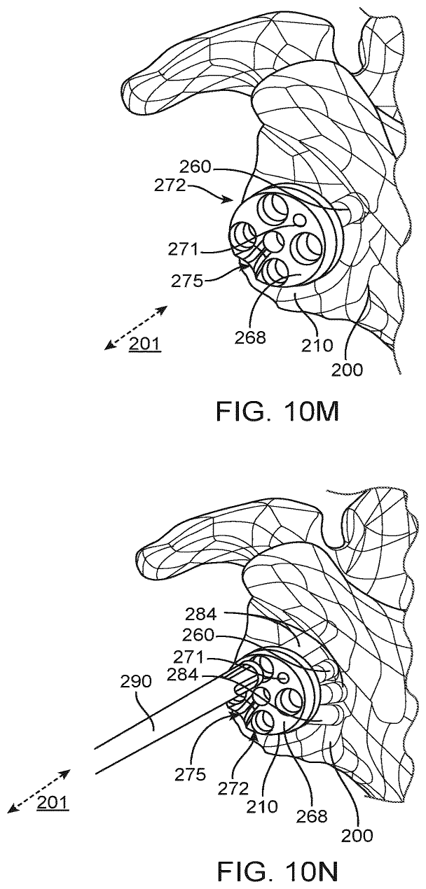

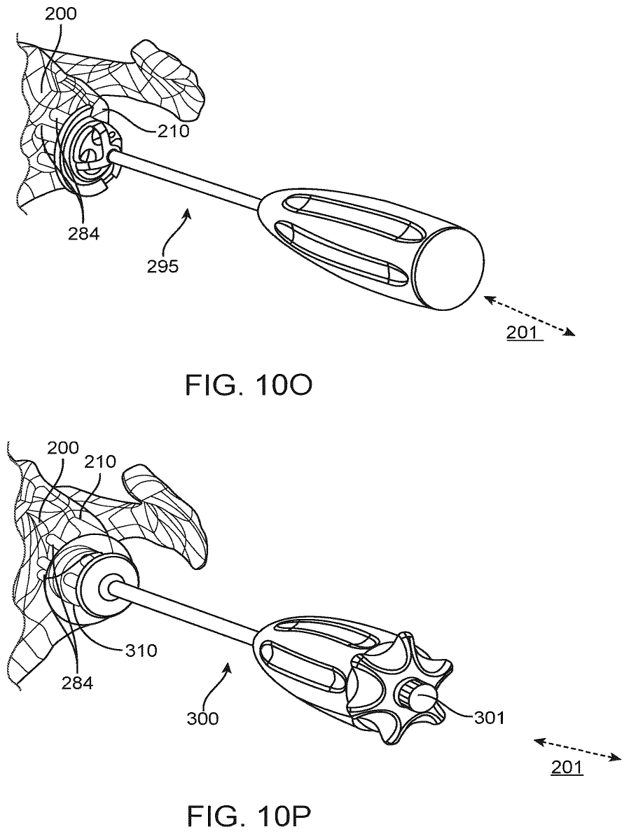

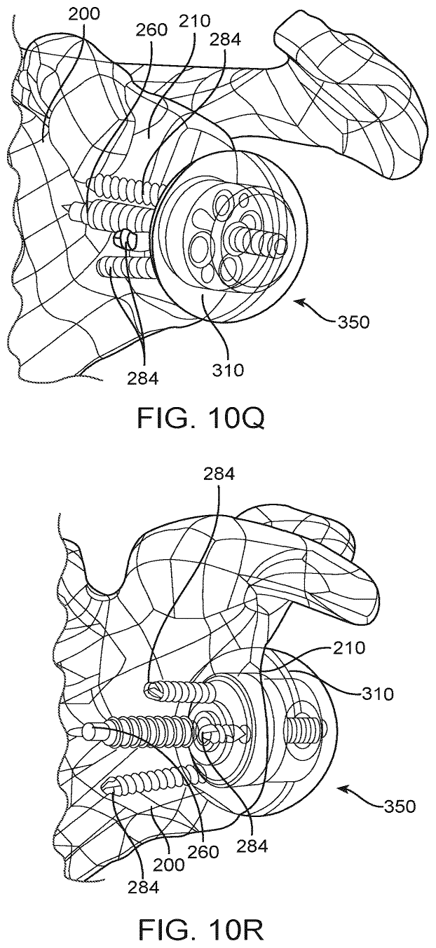

FIGS. 10A-R illustrate methods, systems, and tools used to prepare and place a glenoid implant in a shoulder surgery (e.g., reverse shoulder surgery). In some embodiments, said methods, systems, and tools of the glenoid implant are as described in any one or more of U.S. application Ser. No. 16/817,440 (filed Mar. 12, 2020), U.S. application Ser. No. 17/619,039 (filed Jun. 12, 2020), and U.S. application Ser. No. 17/248,601 (filed Jan. 29, 2021), each of which are incorporated herein by reference in its entirety. In some embodiments, the glenoid implant 350 comprises one or more perimeter screws 284, a glenoid fixation screw 260, and a glenosphere 310. In some embodiments, when placing a glenoid implant 350 in a patient, a working axis a working axis 201 is established that corresponds to a proper angle of insertion (of the glenoid implant) into a glenoid portion of a scapula bone 200 at a glenoid implant site 210. As shown in FIG. 10B, a working axis 201 may be designated by disposing a surgical guide pin 202 into a glenoid implant site 210 along the working axis 201. In some embodiments, a glenoid pin guide alignment device 212 is placed over a glenoid implant site 210 and a surgical guide pin 202 is passed through a glenoid pin guide alignment device bore 219 disposed within a glenoid pin guide alignment device shaft 218 along a working axis 201.

As illustrated in FIG. 10C, for example, a glenoid reamer 224 may be passed over a working axis 201 to ream a glenoid implant site 210. In some embodiments, the glenoid reamer 224 has three teeth (not shown) disposed on the reamer bone facing surface and improve stability and provide minimal micromotion. When the glenoid reamer 224 is removed from the glenoid implant site 210, three rings can be seen on the glenoid implant site 210 as illustrated in FIG. 10D.

As shown in FIG. 10E a pilot hole device 236 is passed over the surgical guide pin 202 and into a glenoid implant site 210. The pilot hole device 236 may create a pilot hole for a screw to anchor the glenoid implant 350. As shown in FIG. 10F, a glenoid depth gauge 240 may be passed into the hole in the glenoid portion of the scapula bone 200 made by the pilot hole device 236 to measure the depth of the pilot hole. As shown in FIGS. 10G and 10H, a glenoid fixation screwdriver is used to screw a glenoid fixation screw 260 into a glenoid implant site 210. In some embodiments, a glenoid fixation screwdriver may have a tapered distal end configured to mate with the proximal end of a glenoid fixation screw 260. In some embodiments, the distal end is a male hexagon taper and the glenoid fixation screw 260 has proximal hexagon shaped receiving end.

In some embodiments, the glenoid fixation screw 260 is a bull nose screw. In some embodiments, the glenoid fixation screw 260 is blunted at the end first entering bone to prevent damage. In some embodiments, 260 has a smooth portion extending approximately 3 mm from the proximal end to a threaded portion. In some embodiments, the glenoid fixation screw 260 is initially inserted proud or at least partially extended from the glenoid implant site 210. In some embodiments, having the glenoid fixation screw 260 at least partially extend from the glenoid implant site 210 facilitates coupling of the glenoid fixation screw 260 with the baseplate 272.

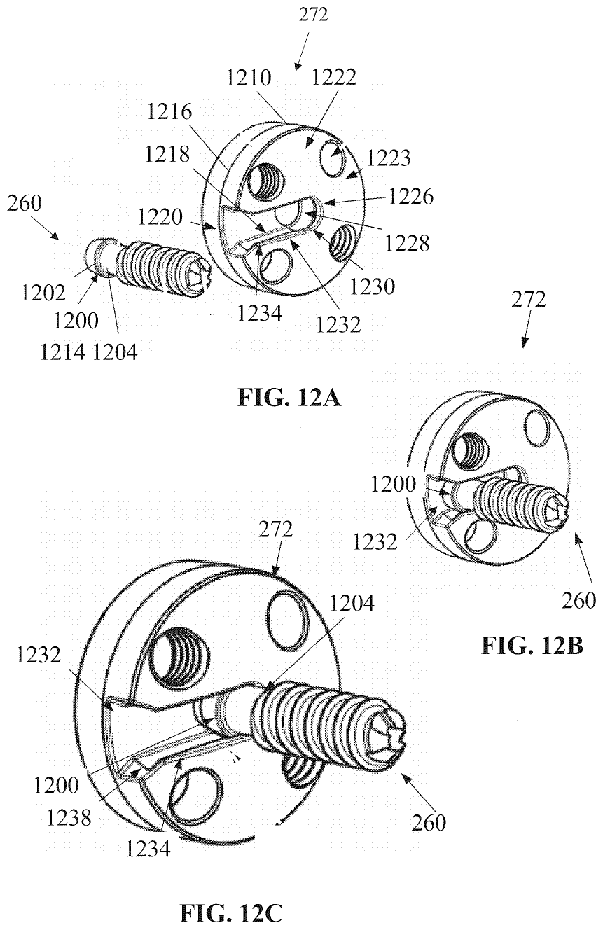

As shown in FIG. 101, the baseplate 272 may be coupled with a glenoid fixation screw 260 by sliding a baseplate fixation screw receiving slot 1232 (see e.g., FIGS. 12A-12C) over the head of a glenoid fixation screw 260. In some embodiments, the baseplate 272 is held by a delivery device 273. As shown in FIG. 10J after the baseplate 272 is coupled with the glenoid fixation screw 260, the glenoid fixation screw 260 may be tightened using a tightening screwdriver 280. In some embodiments, the baseplate 272 has a baseplate dimple 271. In some embodiments, the baseplate dimple 271 is used to align the baseplate glenosphere receiving slot 275 with the head of the glenoid fixation screw 260 head. In some embodiments, the baseplate glenosphere receiving slot 275 is disposed in a portion of the glenosphere facing surface of the baseplate 272 in line with the baseplate dimple 271 along the latitudinal axis of the baseplate. In some embodiments, the baseplate dimple 271 is used to align the baseplate fixation screw receiving slot 1232 with a portion of the glenosphere 310. In some embodiments, the baseplate fixation screw receiving slot 1232 is disposed in a portion of the glenoid facing surface of the baseplate 272 where the radial axis of the slot passes beneath the baseplate dimple 271.

In some embodiments, the baseplate 272 has a rounded bone facing surface to provide optimal angular placement at the glenoid implant site 210, and stability in the case of bone erosion. In some embodiments, the baseplate 272 has at least one handling nubbin 274 at least partially disposed in a baseplate opening. In some embodiments, a delivery device 273 is used to grasp one or more handling nubbin 274 to couple the baseplate 272 to the glenoid fixation screw 260, as shown, for example, in FIG. 101. In some embodiments, the baseplate 272 has four handling nubbins 274 disposed in four openings disposed through the baseplate 272 and arranged symmetrically around the working axis 201 center of the baseplate 272. In some embodiments, the handling nubbins 274 act as guides for drilling pilot holes for perimeter screws 284 as shown for example, in FIGS. 10K and 10L. In some embodiments, the perimeter screw pilot drill 286 may have indications to determine the depth of the pilot hole.

As shown in FIGS. 10M and 10N, for example, once the nubbins 274 are removed a driver 290 may be used to place one or more perimeter screws through the baseplate 272 and into a glenoid implant site 210. In some embodiments, the baseplate 272 is configured to have four perimeter screws 284. In some embodiments, the perimeter screw 284 length are selected based on the pilot hole depth. In some embodiments, one or more pilot screw 284 have different lengths.

As shown in FIGS. 100-10R a glenosphere 310 is coupled to the baseplate 272 using a delivery tool 300. In some embodiments, the glenoid implant site 210 surface is reamed with a surface reamer 295 prior to the glenosphere 310 being coupled to the baseplate 272. In some embodiments, the delivery tool 300 has a shaft threaded into the humerus facing surface of the glenosphere 310. In some embodiments, the bone facing surface of the glenosphere 310 has a coupling member, such as a screw, (not shown) configured to mate with the baseplate 272.

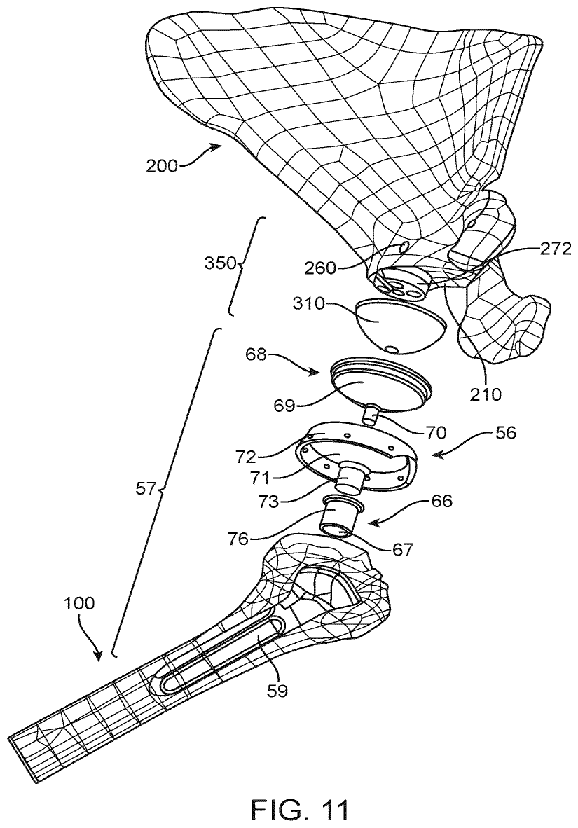

FIG. 11 illustrates an exploded view of a reverse shoulder system and comprising a humeral implant 57 and a glenoid implant 350. In some embodiments, a humeral implant 57 is at least partially disposed in a humerus 100. In some embodiments, the humeral implant 57 comprises a liner 68, an implant tray 56, an adaptor 66, and a stem 59. In some embodiments, the implant tray 56 comprises a tray body portion 71, a tray lip portion 72, and a tray base portion 73. In some embodiments, the adaptor 66 comprises an adaptor wall 76 and an adaptor channel 67. In some embodiments, the stem 59 is at least partially disposed in a humerus 100. In some embodiments, a liner 68, an implant tray 56, an adaptor 66, and a stem 59 are configured to mate together.

In some embodiments, a humeral implant 57 described herein does not comprise a stem 59. For example, as described herein, in some embodiments the implant tray 56 is configured to secure the humeral implant to the humerus via the interface between the tray lip portion 72 and the outer surface 58 of the humeral head excision site. In some embodiments, for such embodiments without a stem, the implant tray 56 maintains a tray base portion 73, and in some cases, maintains an adaptor 66 so as to help reduce any unfilled gaps that may exist between the tray base portion and the excision site. In some embodiments, said excision site only needs to include a cavity to receive the tray base portion and/or the adapter, but not the stem.

In other embodiments of a humeral implant without a stem, the implant tray 56 does not include a tray base portion 73, and thereby the humeral implant does not include an adapter. Accordingly, in such cases, the excision site at the humerus only includes a cavity to receive the tray body portion 71.

In some embodiments, a glenoid implant 350 is configured to be at least partially disposed in a glenoid portion of a scapula bone 200 at a glenoid implant site 210. In some embodiments, a glenoid implant 350 comprises a glenosphere 310, a baseplate 272, and a glenoid fixation screw 260. In some embodiments, the glenosphere 310, the baseplate 272, and the glenoid fixation screw 260 are configured to couple at the glenoid implant site 210.

FIGS. 12A-C illustrate a baseplate 272 and a glenoid fixation screw 260 of the glenoid implant 350 in various stages of coupling. FIG. 1A illustrates the glenoid fixation screw 260 decoupled from a glenoid fixation screw 260.

FIGS. 12A-C illustrate, a baseplate 272 may be placed on a glenoid fixation screw 260, and secured to a glenoid implant site 210, which may be assisted by a delivery device 273 used to place the baseplate 272 over a glenoid fixation screw 260.

Referring to FIGS. 12A-12C, a baseplate 272 may include a body 1210 defining a glenoid bone facing surface 1223 and a glenosphere facing surface 268. The glenoid bone facing surface 1223 may have a surface profile/contour that substantially corresponds to the surface profile/contour of the glenoid implant site. For example, the glenoid bone facing surface 1223 may have a generally convex shape that inversely corresponds to the generally concaved shape of the glenoid implant site.

At least a portion of a glenosphere facing surface 268 of the baseplate 272 may be configured to be coupled to a glenoid implant 350. The glenosphere facing surface 268 may have a generally convex shape configured to be received in at least a glenoid implant 350.

With respect to FIGS. 12A-12C, an exemplary bone facing surface of the baseplate 272 with a glenoid fixation screw 260 is shown. In some embodiments, the glenoid bone facing surface 1223 of the baseplate 272 also includes a baseplate channel 1232, for example, extending from an outer side surface/periphery 1216 of the body 1210. The baseplate channel 1232 is configured to receive a fixation screw head 1200 and a portion of the shank 1204 of the glenoid fixation screw 260 (e.g., as generally best illustrated in FIGS. 12A-12C). In particular, the baseplate channel 1232 may have a cross-section generally corresponding to the cross-section of the fixation screw head 1200 and a portion of the shank 1204 such that the fixation screw head 1200 and a portion of the shank 1204 can be received through an entrance 1220 of the baseplate channel 1232 and enter into the baseplate channel 1232, but once inside the baseplate channel 1232, cannot be removed from the baseplate channel 1232 other than through the entrance 1220 (due to at least a portion of the head 1200 being disposed within a slot 1234 in the baseplate channel 1232, as described herein).

The baseplate channel 1232 may extend from the outer side surface/periphery 1216 of the body 1210 to a central region (e.g., a center) of baseplate 272. In at least one example, the baseplate channel 1232 may be formed at least in part in a glenoid bone facing surface 1223 of the body 1210. The lateral entrance 1220 may be formed in the outer side surface/periphery 1216 of the body 1210 while the slot/open region 1234 of the baseplate channel 1232 may be formed by the glenoid bone facing surface 1223. The entrance 1220 may have a larger cross-section than the fixation screw head 1200, and may be tapered, to facilitate alignment and advancement of the fixation screw head 1200 through the entrance 1220 and into the baseplate channel 1232. The taper may include a taper that increases closer to the glenoid bone facing surface 1223 and/or a taper that decreases closer to the glenoid bone facing surface 1223, and each taper may correspond to the taper of the fixation screw head 1200. The baseplate channel 1232 may include interior surfaces 1218 forming an undercut (e.g., having a concaved profile). In one example, at least a portion of the interior surfaces 1218 (e.g., the bottom portion) generally corresponds to the cross-section of the fixation screw head 1200 (e.g., the taper of the fixation screw head 1200). The interior surfaces 1218 of the baseplate channel 1232 may also be configured to facilitate alignment and advancement of the fixation screw head 1200 through the baseplate channel 1232, e.g., as generally illustrated in FIGS. 12A-12C. The slot/open region 1234 of the baseplate channel 1232 may generally correspond to the cross-section of the shank 1204 of the glenoid fixation screw 260.

A distal end region 1226 of the baseplate channel 1232 includes a center anchor receptacle 1228 (e.g., recess/pocket). The center anchor receptacle 1228 is configured to receive at least a portion of the fixation screw head 1200 of the glenoid fixation screw 260. In at least one example, the fixation screw head 1200 may include an anchor engagement surface 1214 configured to engage with a corresponding baseplate engagement surface 1238 of the recess/pocket 1230. For example, the anchor engagement surface 1214 may include a shoulder 1202 having a cross-section (e.g., a diameter) that substantially corresponds to the cross-section (e.g., a diameter) of the baseplate engagement surface 1238 of the center anchor receptacle 1228. At least one embodiment, the baseplate engagement surface 1238 of the center anchor receptacle 1228 may form a generally cylindrical recess/pocket. Alternatively (or in addition), the anchor engagement surface 1200 may include a taper that substantially corresponds to a taper of the baseplate engagement surface 1238 of the center anchor receptacle 1228 to form a tapered undercut interference connection, which should also be understood as a positive mechanical engagement connection.

With reference now to FIGS. 12A-12C, once the glenoid fixation screw 260 has been secured to the glenoid implant site 210, the fixation screw head 1200 of the glenoid fixation screw 260 may be advanced through the entrance 1220 (e.g., FIG. 12B) and into the baseplate channel 1232 until the fixation screw head 1200 is proximate the center anchor receptacle 1228 (e.g., FIG. 12C). Once the fixation screw head 1200 is proximate the center anchor receptacle 1228, the glenoid fixation screw 260 may be secured to the baseplate 272. Delivery device 273 may be used to hold the baseplate 272. Once the fixation screw head 1200 of the glenoid fixation screw 260 is mechanically locked in the baseplate channel 1232 of the baseplate 272 as described above, which inhibits separation of the glenoid fixation screw 260 and the baseplate 272 along the working axis, the delivery device 273 may be removed. In some embodiments, the baseplate 272 is secured to the glenoid implant site 210.

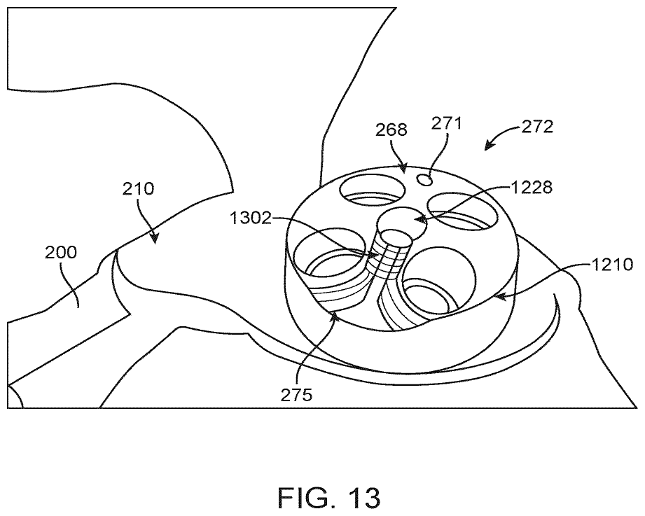

FIG. 13 illustrates the baseplate 272 according the one embodiment of the present disclosure. The glenosphere facing surface 268 of a baseplate 272 of this embodiment includes an elongated channel 1302 extending from a fixation element in the form of a center post receptacle 1228 (e.g., recess/hole) to the periphery 1210 of the baseplate 272, and a baseplate glenosphere receiving slot 275 near the outer run of the channel 1302. The baseplate glenosphere receiving slot 275 may be generally deeper than the channel 1302 and may be flared toward the outer periphery of the baseplate 272, as shown.

In some embodiments, the baseplate 272 is configured to receive the glenoid fixation screw 260 at least partially disposed in a glenoid implant site 210. In some embodiments, the baseplate 272 receives the glenoid fixation screw 260 by sliding the baseplate 272 over the fixation screw head 1200 of the glenoid fixation screw 260 and along the baseplate channel 1232. In some embodiments, the glenosphere facing surface 268 of baseplate 272 comprises a baseplate dimple 271 disposed on the other side of the center post receptacle 1228 from a baseplate glenosphere receiving slot 275 in line along the latitudinal axis of the baseplate 272. In some embodiments, a baseplate glenosphere receiving slot 275 disposed in the glenosphere facing surface 268 is configured to receive a portion of glenosphere 310 by sliding the portion of the glenosphere 310 into and along the baseplate glenosphere receiving slot 275. In some embodiments, the baseplate glenosphere receiving slot 275 is configured to be in line with the baseplate dimple 271 and baseplate channel 1232 along the latitudinal axis of a baseplate 272.

In some embodiments, the baseplate 272 is a smaller size than traditional baseplates used in shoulder revision surgeries, because the baseplate 272 couples to the glenoid fixation screw 260 by the sliding mechanism of coupling a fixation screw head 1200 and a baseplate channel 1232. Traditional baseplates may necessitate a larger size than the baseplate 272 disclosed herein, because they are attached to the glenoid implant site by screwing a glenoid fixation screw through a center post receptacle. Smaller baseplates may be advantageous in shoulder surgeries, because they need smaller implant sites and less reaming of the glenoid, thus preserving native bone.

As used in this specification and the appended claims, the singular forms “a,” “an,” and “the” include plural references unless the context clearly dictates otherwise. As used in this specification and the claims, unless otherwise stated, the term “about,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, or +/−15%, depending on the embodiment. As a non-limiting example, about 100 meters represents a range of 95 meters to 105 meters, 90 meters to 110 meters, or 85 meters to 115 meters depending on the embodiments. The term “substantially” refers to less than or equal to +/−1%, +/−2%, +/−3%, +/−4%, +/−5%, +/−6%, +/−7%, +/−8%, +/−9%, +/−10%, +/−11%, +/−12%, +/−14%, or +/−15% variation. As a non-limiting example, substantially parallel represents a range of −1 to 1 degree difference, −5 to 5 degree difference, or −15 degrees to 15 degrees of difference from being parallel, depending on the embodiments.

1. A system for performing a reverse shoulder replacement surgery on a subject, the system comprising:
a tray having a body, a lip extending from a proximal portion thereof, and a tray base portion extending from a distal portion of the body, the body configured to be at least partially received within a concave inner surface of an implant site in a humerus of the subject, the lip configured to engage an outer surface of the implant site, thereby coupling the tray to the humerus;
a liner configured to be at least partially disposed within the tray;
a stem configured to be disposed at least partially within the humerus, the stem comprising an open cavity at a proximal portion thereof configured to receive at least a portion of the tray base portion; and
an adapter disposed between the stem and the tray and configured to at least partially fill a spacing between an inner surface within the cavity of the stem and the tray base portion.

2. The system of claim 1, wherein the inner surface of the implant site comprises an opening for the tray base portion to extend therethrough towards the stem.

3. The system of claim 1, wherein the adapter comprises a foundation portion and a wall extending therefrom, wherein the foundation portion and the wall define a hollow portion therein configured to at least partially receive the tray base portion.

4. The system of claim 1, wherein the adapter is configured with a cylindrical or substantially cylindrical shape.

5. The system of claim 3, wherein a desired size of the adapter corresponds to the spacing between the inner surface within the stem cavity and the tray base portion, the desired size of the adapter comprising a thickness of the foundation portion, a length of the adapter, a width of the adapter, and/or a thickness of the wall.

6. The system of claim 5, wherein the length of the adapter corresponds to a sum of the foundation portion thickness and a length of the wall.

7. The system of claim 5, wherein the width of the adapter comprises a sum of the width of the hollow portion and a thickness of the wall.

8. The system of claim 5, wherein the width of the adapter corresponds to a diameter.

9. The system of claim 5, further comprising an adapter offset tool configured to determine the spacing between the inner surface within the cavity and the tray base portion when inserted therein, so as to determine the desired size of the adapter.

10. The system of claim 9, wherein the adapter offset tool comprises an outer shaft, and an inner shaft configured to move telescopically within a channel of the outer shaft.

11. The system of claim 10, wherein when a distal portion of the inner shaft is inserted within the stem cavity and abuts the inner surface thereof, a depth of a proximal portion of the inner shaft located within the outer shaft channel corresponds to a spacing between the inner surface within the stem cavity and the tray base portion.

12. The system of claim 11, wherein an indicator on an outer surface of the proximal portion of the inner shaft is visible through a window of the outer shaft, thereby indicating the depth of the proximal portion within the outer shaft channel.

13. The system of claim 9, wherein the offset tool further comprises a trial tray configured to be received by the concave inner surface of the implant site, the trial tray comprising an inner surface at a proximal portion thereof configured to abut with the outer shaft, thereby enabling the inner shaft to extend from the outer shaft through an opening in the trial tray towards the stem.

14. The system of claim 1, wherein the lip and the body of the tray are configured to be secured to the implant site via friction fit.

15. The system of claim 1, wherein the tray is detachably coupled to the implant site.

16. The system of claim 1, wherein the stem comprises a distal portion extending from the proximal portion and configured to be disposed within the humerus bone without affixation thereto, such that the stem is secured within the humerus bone via the coupling between the tray and the implant site.

17. The system of claim 1, further comprising a glenoid implant disposed in an implant site at a glenoid of the subject, the glenoid implant comprising a head implant configured to articulate within an inner surface of the liner.

18. The system of claim 1, wherein the head implant comprises a glenosphere.

19.-26. (canceled)

27. An offset tool for determining a desired size of an adapter for use with a humeral implant, the offset tool comprising:
a trial tray configured to be coupled with a humeral implant site, the trial tray comprising an opening at a distal portion thereof;
an outer shaft having a distal end and a proximal end, the distal end having an aperture to a channel within the outer shaft, the distal end having a dimension larger than a dimension of the opening such that the distal end is configured to abut the trial tray at a proximal surface thereof, and
an inner shaft having an inner shaft proximal portion and an inner shaft distal portion, the inner shaft proximal portion configured to move telescopically within the channel of the outer shaft, such that the inner shaft is configured to extend from the outer shaft and through the opening towards a stem disposed in a humeral stem;
wherein, when the inner shaft distal portion abuts an inner surface of a cavity of the stem and the outer shaft distal end abuts the trial tray coupled to the implant site, the desired size of the adapter corresponds with a depth therebetween.

28. The offset tool of claim 27 further comprising, at least one groove recessed in the distal end of the distal portion.

29. The offset tool of claim 27 further comprising, a window disposed through the outer shaft.

30. The offset tool of claim 29 further comprising, an indicator on an outer surface of the proximal portion of the inner shaft that is configured to be visible through the window of the outer shaft, thereby indicating the depth of the proximal portion within the outer shaft channel.

31. The offset tool of claim 29, further comprising, offset markings disposed around the window.
